(19) United States
(12) Patent Application Publication
(10) Pub. No.: US 2006/0276520 A1
(43) Pub. Date: Dec. 7, 2006

(54) Rhodanine derivatives and pharmaceutical compositions containing them

(75) Inventors: Rajinder Singh, Belmont, CA (US); Usha V. Ramesh, Cupertino, CA (US); Dane Goff, Redwood City, CA (US); Sarkiz D. Issakani, San Jose, CA (US); Jianing Huang, Foster City, CA (US); Donald G. Payan, Hillsborough, CA (US); Jeffrey Clough, Redwood City, CA (US)

Correspondence Address: MCDONNELL BOEHNEN HULBERT & BERGHOFF LLP, 300 S. WACKER DRIVE, 32ND FLOOR, CHICAGO, IL, 60606, US

(73) Assignee: Rigel Pharmaceuticals, Inc., South San Francisco (US)

(21) Appl. No.: 10/534,919

(22) PCT Filed: Nov. 13, 2003
(86) PCT No.: PCT/US03/36747

§ 371 Date: Apr. 14, 2006

Related U.S. Application Data
(60) Provisional application No. 60/426,280, filed on Nov. 13, 2002.
(60) Provisional application No. 60/514,951, filed on Oct. 28, 2003.

(51) Int. Cl. A61K 31/427; A61K 31/426; A61K 31/422; A61K 31/421; A61K 31/4178; C07D 417/02; C07D 413/02; C07D 405/02

(52) U.S. Cl. 514/369; 548/183; 548/227; 548/315.4; 548/316.7; 514/376; 514/389

(57) ABSTRACT

This invention describes rhodanine derivatives and pharmaceutical compositions useful as inhibitors of ubiquitination. The compounds and compositions of the invention are useful as inhibitors of the biochemical pathways of organisms in which ubiquitination is involved. In particular, the compounds and compositions are useful for treating cell proliferative diseases such as cancers.

This application claims priority to provisional application MBHB Attorney Docket No. MBHB-03-004-A, filed Oct. 28, 2003, and provisional application U.S. Ser. No. 60/426,280, filed Nov. 13, 2002.

### BACKGROUND OF THE INVENTION

1. Field of the Invention

This invention is in the field of ubiquitin ligation and inhibitors of the ubiquitination pathway. Additionally, this invention is in the field of treating diseases or conditions associated with ubiquitination.

2. Summary of the Related Art

Ubiquitin is a 76 amino acid protein present throughout the eukaryotic kingdom. It is a highly conserved protein and is essentially the identical protein in diverse organisms ranging from humans to yeasts to fruit flies. In eukaryotes, ubiquitin is the key component of the ATP-dependent pathway for protein degradation. Proteins slated for degradation are covalently linked to ubiquitin via an ATP-dependent process catalyzed by three separate enzymes.

The ubiquitination of these target proteins is known to be mediated by the enzymatic activity of three ubiquitin agents. Ubiquitin is first activated in an ATP-dependent manner by a ubiquitin activating agent, for example, an E1. The C-terminus of a ubiquitin forms a high energy thioester bond with the ubiquitin activating agent. The ubiquitin is then transferred to a ubiquitin conjugating agent, for example, an E2 (also called ubiquitin moiety carrier protein), also linked to this second ubiquitin agent via a thiolester bond. The ubiquitin is finally linked to its target protein (e.g. substrate) to form a terminal isopeptide bond under the guidance of a ubiquitin ligating agent, for example, an E3. In this process, monomers or oligomers of ubiquitin are attached to the target protein. On the target protein, each ubiquitin is covalently ligated to the next ubiquitin through the activity of a ubiquitin ligating agent to form polymers of ubiquitin.

The enzymatic components of the ubiquitination pathway have received considerable attention (for a review, see Weissman, Nature Reviews 2:169-178 (2001); see also Wong et al., Drug Discov. Today 8(16), 46-54 (2003)). The members of the E1 ubiquitin activating agents and E2 ubiquitin conjugating agents are structurally related and well characterized enzymes. There are numerous species of E2 ubiquitin conjugating agents, some of which act in preferred pairs with specific E3 ubiquitin ligating agents to confer specificity for different target proteins. While the nomenclature for the E2 ubiquitin conjugating agents is not standardized across species, investigators in the field have addressed this issue and the skilled artisan can readily identify various E2 ubiquitin conjugating agents, as well as species homologues (See Haas and Siepmann, FASEB J. 11:1257-1268 (1997)).

Furthermore, ubiquitin agents, such as the ubiquitin activating agents, ubiquitin conjugating agents, and ubiquitin ligating agents, are key determinants of the ubiquitin-mediated proteolytic pathway that results in the degradation of targeted proteins and regulation of cellular processes. Consequently, agents that modulate the activity of such ubiquitin agents may be used to up-regulate or down-regulate specific molecules involved in cellular signal transduction. Disease processes can be treated by such up- or down regulation of signal transducers to enhance or dampen specific cellular responses. This principle has been used in the design of a number of therapeutics, including phosphodiesterase inhibitors for airway disease and vascular insufficiency, kinase inhibitors for malignant transformation and proteasome inhibitors for inflammatory conditions such as arthritis.

There is a need for inhibitors of ubiquitination that can alter the ATP-dependent ubiquitination of proteins. Inhibition of ubiquitination can regulate the degradation of proteins in ways that assist in treating various disorders. Inhibitors of ubiquitin ligases may also help in treating infectious diseases such as bacterial and viral infections that depend on the cellular biochemical machinery.

Due to the importance of ubiquitin-mediated proteolysis in cellular process, for example cell cycle regulation, there is also a need for a fast and simple means for identifying the physiological role of ubiquitin agents that are catalytic components of this enzymatic pathway, and for identifying which ubiquitin agents are involved in various regulatory pathways. Pray et al., Drug Resist. Update 2(2), 249-258 (2002). Thus, an object of the present invention is to provide compounds, compositions and methods of assaying for the physiological role of ubiquitin agents, and for providing methods for determining which ubiquitin agents are involved together in a variety of different physiological pathways.

### BRIEF DESCRIPTION OF THE INVENTION

The invention comprises compounds, pharmaceutical compositions of the compounds for inhibiting ubiquitination. The pharmaceutical compositions can be used in treating various conditions where ubiquitination is involved. They can also be used as research tools to study the role of ubiquitin in various natural and pathological processes.

In a first aspect, the invention comprises compounds that inhibit ubiquitination of target proteins.

In a second aspect, the invention comprises a pharmaceutical composition comprising an inhibitor of ubiquitination according to the invention and a pharmaceutically acceptable carrier, excipient, or diluent.

In a third aspect, the invention comprises methods of inhibiting ubiquitination in a cell, comprising contacting a cell in which inhibition of ubiquitination is desired with a pharmaceutical composition comprising a ubiquitin agent inhibitor according to the invention.

In a fourth aspect, the invention provides methods for treating cell proliferative diseases or conditions, comprising administering to a patient in need thereof a pharmaceutical composition comprising an effective amount of a ubiquitin agent inhibitor according to the invention.

The foregoing only summarizes certain aspects of the invention and is not intended to be limiting in nature. These aspects and other aspects and embodiments are described more fully below. All patent applications and publications of any sort referred to in this specification are hereby incorporated by reference in their entirety. In the event of a discrepancy between the express disclosure of this specification and a patent application or publication incorporated by reference, the express disclosure of this specification shall control.

### DETAILED DESCRIPTION OF THE INVENTION

The first aspect of the invention comprises compounds having the formula 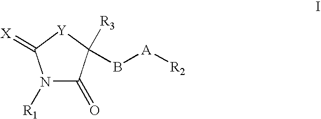
or pharmaceutically acceptable salts thereof, wherein
A is aryl or heteroaryl;
B is C₁-C₆ alkyl or C₂-C₆ alkenyl;
X is sulfur, oxygen, ═CR₄R₅, ═NR₄, ═NC(O)R₄, or ═NSO₂R₄;
Y is sulfur, oxygen, —C(R₄)(R₅)—, —N(R₄)—, —NC(O)(R₄), —NSO₂(R₄)—, —S(O)₂—, or —S(O)—;
R₁ is —H, —NH₂, C₁-C₆ alkyl, C₁-C₂ alkenyl, C₁-C₆ alkyl-S—C₁-C₆ alkyl, C₀-C₆ alky-aryl, C₀-C₆ alkyl-C(O)OR₆, C₀-C₆ alkyl-heteroaryl, C₀-C₆ alkyl-heterocyclyl, C₀-C₆ alkyl-carbocyclyl, —NH—SO₂-aryl, —C₀-C₆ alkyl-C(O)NR₆R₇, —C₀-C₆ alkyl-C(S)NR₆R₇, C₀-C₆ alky-heteroaryl-aryl, —NHC(O)-aryl, C₀-C₆ alkyl-C(O)NH—C₀-C₆ alkyl-C(O)—O—R₆, C₀-C₆ alkyl-C(O)—NH—C₀-C₆ alkyl-aryl, C₀-C₆ alkyl-C(O)—NH—C₀-C₆ alkyl-heteroaryl, C₀-C₆ alkyl-C(O)—NH—C₀-C₆ alkyl-heterocyclyl, C₀-C₆ alkyl-C(O)—NH—C₀-C₆ alkyl-carbocyclyl, —SO₂—R₆, C(O)—R₆, or —C(O)—OR₆, wherein each one of the alkyl, aryl, heteroaryl, heterocyclic and carbocyclyl are optionally substituted with one or more R₅;
R₂ is —H, halogen, C₁-C₆ alkyl, C₀-C₆ alky-aryl, —NO₂, C₀-C₆ alkyl-C(O)—OR₆, C₀-C₆ alkyl-heteroaryl, C₀-C₆ alkyl-heterocyclyl, C₀-C₆ alkyl-carbocyclyl, —N(R₆)—C(O)NR₆R₇, —NHSO₂-aryl, C₀-C₆ alky-heteroaryl-aryl, or —C(O)—R₆, wherein each one of the aryl, heteroaryl, heterocyclic and carbocyclyl are optionally substituted with one or more R₄;
R₃ is —H, C₁-C₆ alkyl or C₂-C₆ alkenyl; or
R₃ and B together with the carbon atom to which they are attached form an alkenyl or a spirocyclic ring;
R₄ is halogen, oxo, —C(O)OR₆, —NO₂, C₁-C₆ alkyl optionally substituted with halo, —C₁-C₆ alkoxy optionally substituted with halo, —CH₃, —SO₂NH₂, or —C(O)—OR₆;
R₅ is halogen, oxo, C₁-C₆ alkoxy, C₁-C₆ alkyl, C₀-C₆ alkyl-aryl, —NO₂, di(C₁-C₆ alkyl)amino, —CF₃, —OH, —SO₂NH₂, or —C(O)OR₆; and
R₆ and R₇ are independently —H, halogen, C₁-C₆ alkoxy, C₁-C₆ alkyl, C₂-C₆ alkenyl, aryl, di(C₁-C₆ alkyl)amino, —CF₃, —OH, or —C(O)—OR₆.

In a preferred embodiment, the invention also comprises compounds of paragraph [0015] having the formula

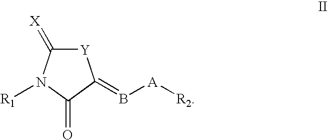

In another preferred embodiment, the invention further comprises compounds of paragraph [0016] of the formula

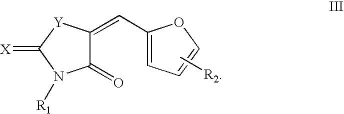

In a preferred embodiment of the invention the compounds of formula III are compounds wherein R₁ is —H, C₁-C₆ alkyl, C₁-C₂ alkenyl, C₀-C₆ alky-aryl, C₀-C₆ alkyl-C(O)OR₆, C₀-C₆ alkyl-heteroaryl, C₀-C₆ alkyl-heterocyclyl, C₀-C₆ alkyl-carbocyclyl or C₀-C₆ alky-heteroaryl-aryl, and R₂ is —H, halogen, C₁-C₆ alkyl or C₀-C₆ alky-aryl. More preferably, R₁ is —H, C₁-C₆ alkyl, C₁-C₂ alkenyl, C₀-C₆ alky-aryl, or C₀-C₆ alkyl-C(O)OR₆ and R₂ is C₀-C₆ alky-aryl. Even more preferably, R₁ is —H, allyl, phenyl or benzyl, and R₂ is phenyl.

In another preferred embodiment, the invention also comprises compounds of paragraph

[∫]of the formula

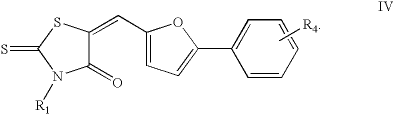

Preferably, the compounds of formula IV are compounds wherein R₁ is —H, C₁-C₆ alkyl, C₁-C₂ alkenyl, C₀-C₆ alky-aryl-C₀-C₆ alkyl-C(O)OR₆, C₀-C₆ alkyl-heteroaryl, C₀-C₆ alkyl-heterocyclyl, C₀-C₆ alkyl-carbocyclyl or C₀-C₆ alky-heteroaryl-aryl, and R₄ is halogen, oxo, —NO₂, C₁-C₆ alkyl, C₁-C₆ alkoxy, —CF₃, —SO₂NH₂ or —C(O)—OR₆. More preferably, R₁ is —H, C₁-C₆ alkyl, C₁-C₂ alkenyl, C₀-C₆ alky-aryl or C₀-C₆ alkyl-C(O)OR₆, and R₄ is halogen, —NO₂, C₁-C₆ alkyl, —C₁-C₆ alkoxy, —CF₃, —SO₂NH₂ or —C(O)—OR₆. Even more preferably, R₁ is —H, allyl, phenyl or benzyl, and R₄ is chloro, bromo, fluoro, —NO₂, —OCH₃, —CF₃ or —C(O)—OH.

In another embodiment, the invention comprises compounds of paragraph [0015] or [0016] that are not also compounds of any of paragraphs [0017]-[0020].

The second aspect of the invention comprises pharmaceutical compositions comprising a pharmaceutically acceptable carrier, diluent or excipient, and a compound of formula I as described in any one of paragraphs [0015]-[0021].

The compounds and pharmaceutical compositions of the invention are useful as inhibitors of ubiquitination because they inhibit ubiquitin agents that are the enzymes involved in the ubiquitination pathway. Specifically, the compounds and compositions of the invention inhibit the ubiquitin ligating activity of the E3 enzyme. Inhibition of the E3 enzyme also decreases the upstream functions of the E1 (ubiquitin activation with ATP) and E2 (transfer of activated ubiquitin to E3) enzymes. Accordingly, the compounds and compositions of the invention are useful for the inhibition of ubiquitination in a cell or in a patient suffering from a disease or condition that involves ubiquitination.

Thus, the third aspect of the invention comprises methods of inhibiting ubiquitination in a cell comprising contacting a cell in which inhibition of ubiquitination is desired with a compound or pharmaceutical composition comprising a ubiquitin agent inhibitor according to the invention.

The fourth aspect of the invention comprises methods for treating cell proliferative diseases or conditions, comprising administering to a patient in need thereof a pharmaceutical composition comprising an effective amount of a ubiquitin agent inhibitor according to the invention. For example, diseases and conditions that can be treated are all types of cancers and conditions related to cancers. However, any disease or condition in which ubiquitination is a component can be treated with the compounds and pharmaceutical compositions of the invention.

The table below illustrates certain preferred embodiments of the first aspect of the invention. We have found that the compounds listed in the table are useful as inhibitors of ubiquitinization, as described more fully below, and, accordingly, useful as anti-cancer agents.

| Cpd | Structure | Name |
|---|---|---|
| 1 | 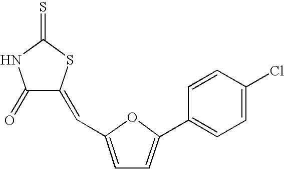 | (5Z)-5-{[5-(4-chlorophenyl)-2-furyl]methylene}-2- thioxo-1,3-thiazolidin-4-one |
| 2 | 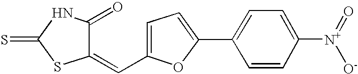 | (5E)-5-{[5-(4-nitrophenyl)-2-furyl]methylene}-2- thioxo-1,3-thiazolidin-4-one |
| 3 | 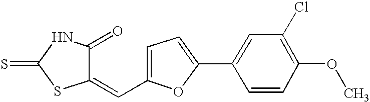 | (5E)-5-{[5-(3-chloro-4-methoxyphenyl)-2- furyl]methylene}-2-thioxo-1,3-thiazolidin-4-one |
| 4 | 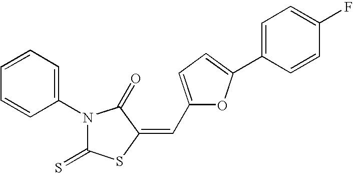 | (5E)-5-{[5-(4-fluorophenyl)-2-furyl]methylene}-3- phenyl-2-thioxo-1,3-thiazolidin-4-one |
| 5 | 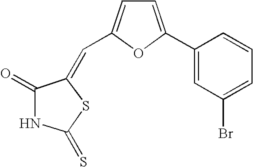 | (5Z)-5-{[5-(3-bromophenyl)-2-furyl]methylene}-2- thioxo-1,3-thiazolidin-4-one |
| 6 | 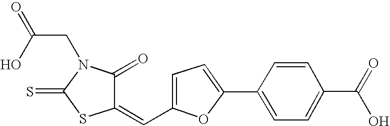 | 4-(5-{(E)-[3-(carboxymethyl)-4-oxo-2-thioxo-1,3- thiazolidin-5-ylidene]methyl}-2-furyl)benzoic acid |
| 7 | 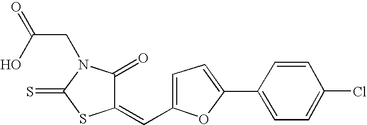 | ((5E)-5-{[5-(4-chlorophenyl)-2-furyl]methylene}-4- oxo-2-thioxo-1,3-thiazolidin-3-yl)acetic acid |
| 8 | 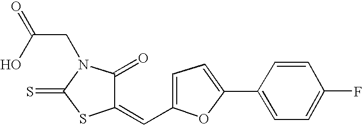 | ((5E)-5-{[5-(4-fluorophenyl)-2-furyl]methylene}-4- oxo-2-thioxo-1,3-thiazolidin-3-yl)acetic acid |
| 9 | 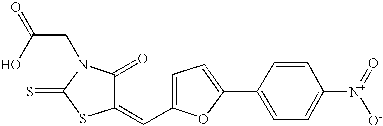 | ((5E)-5-{[5-(4-nitrophenyl)-2-furyl]methylene}-4- oxo-2-thioxo-1,3-thiazolidin-3-yl)acetic acid |
| 10 | 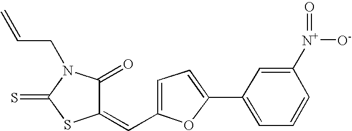 | (5E)-3-allyl-5-{[5-(3-nitrophenyl)-2- furyl]methylene}-2-thioxo-1,3-thiazolidin-4-one |
| 11 | 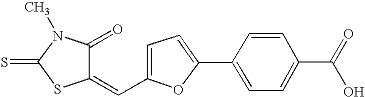 | 4-{5-[(E)-(3-methyl-4-oxo-2-thioxo-1,3-thiazolidin- 5-ylidene)methyl]-2-furyl}benzoic acid |
| 12 | 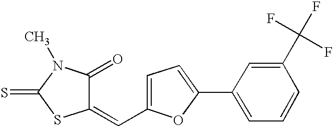 | (5E)-3-methyl-2-thioxo-5-({5-[3- (trifluoromethyl)phenyl]-2-furyl}methylene)-1,3- thiazolidin-4-one |
| 13 | 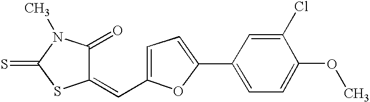 | (5E)-5-{[5-(3-chloro-4-methoxyphenyl)-2- furyl]methylene}-3-methyl-2-thioxo-1,3-thiazolidin- 4-one |
| 14 | 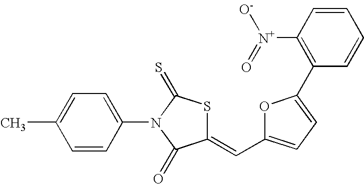 | (5Z)-3-(4-methylphenyl)-5-{[5-(2-nitrophenyl)-2- furyl]methylene}-2-thioxo-1,3-thiazolidin-4-one |
| 15 | 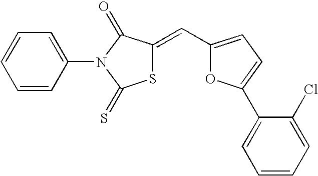 | (5Z)-5-{[5-(2-chlorophenyl)-2-furyl]methylene}-3- phenyl-2-thioxo-1,3-thiazolidin-4-one |
| 16 | 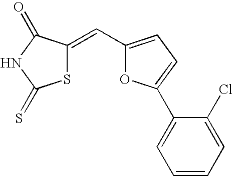 | (5Z)-5-{[5-(2-chlorophenyl)-2-furyl]methylene}-2- thioxo-1,3-thiazolidin-4-one |
| 17 | 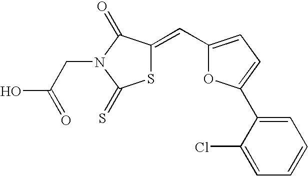 | ((5Z)-5-{[5-(2-chlorophenyl)-2-furyl]methylene}-4- oxo-2-thioxo-1,3-thiazolidin-3-yl)acetic acid |
| 18 | 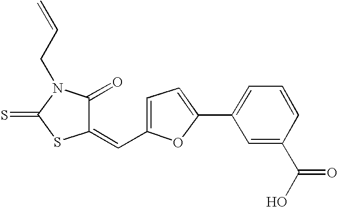 | 3-{5-[(E)-(3-allyl-4-oxo-2-thioxo-1,3-thiazolidin-5- ylidene)methyl]-2-furyl}benzoic acid |
| 19 | 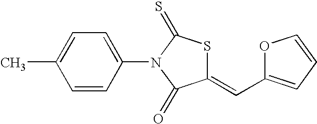 | (5Z)-5-(2-furylmethylene)-3-(4-methylphenyl)-2- thioxo-1,3-thiazolidin-4-one |
| 20 | 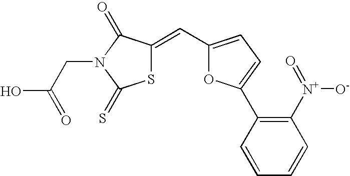 | ((5Z)-5-{[5-(2-nitrophenyl)-2-furyl]methylene}-4- oxo-2-thioxo-1,3-thiazolidin-3-yl)acetic acid |
| 21 | 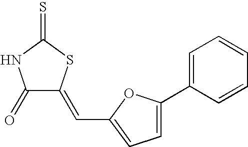 | (5Z)-5-[(5-phenyl-2-furyl)methylene]-2-thioxo-1,3- thiazolidin-4-one |
| 22 | 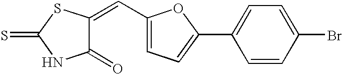 | (5E)-5-{[5-(4-bromophenyl)-2-furyl]methylene}-2- thioxo-1,3-thiazolidin-4-one |
| 23 | 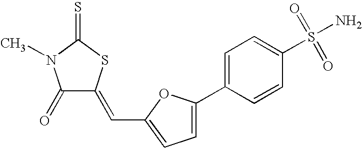 | 4-{5-[(Z)-(3-methyl-4-oxo-2-thioxo-1,3-thiazolidin-5- ylidene)methyl]-2-furyl}benzenezulffonamide |
| 24 | 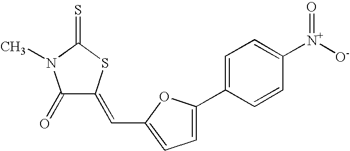 | (5Z)-3-methyl-5-{[5-(4-nitrophenyl)-2- furyl]methylene}-2-thioxo-1,3-thiazolidin-4-one |
| 25 | 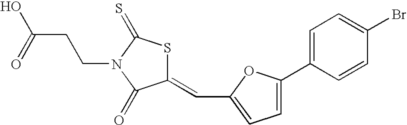 | 3-((5Z)-5-{[5-(4-bromophenyl)-2-furyl]methylene}- 4-oxo-2-thioxo-1,3-thiazolidin-3-yl)propanoic acid |
| 26 | 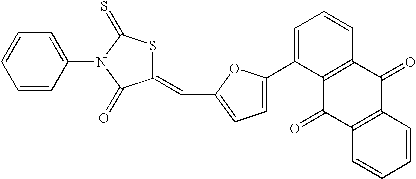 | 1-{5-[(Z)-(4-oxo-3-phenyl-2-thioxo-1,3-thiazolidin-5- ylidene)methyl]-2-furyl}anthra-9,10-quinone |
| 27 | 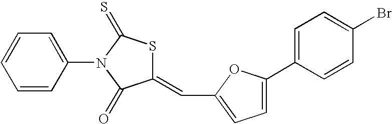 | (5Z)-5-{[5-(4-bromophenyl)-2-furyl]methylene}-3- phenyl-2-thioxo-1,3-thiazolidin-4-one |
| 28 | 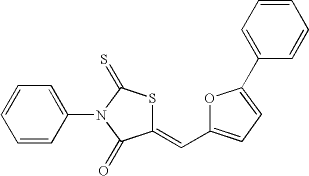 | (5Z)-3-phenyl-5-[(5-phenyl-2-furyl)methylene]-2- thioxo-1,3-thiazolidin-4-one |
| 29 | 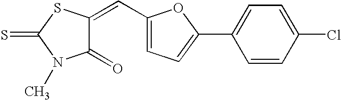 | (5E)-5-{[5-(4-chlorophenyl)-2-furyl]methylene}-3- methyl-2-thioxo-1,3-thiazolidin-4-one |
| 30 | 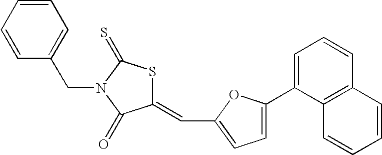 | (5Z)-3-benzyl-5-{[5-(1-nephthyl)-2- furyl]methylene}-2-thioxo-1,3-thiazolidin-4-one |
| 31 | 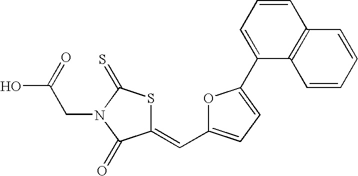 | ((5Z)-5-{[5-(1-naphthyl)-2-furyl]methylene}-4-oxo-2- thioxo-1,3-thiazolidin-3-yl)acetic acid |
| 32 | 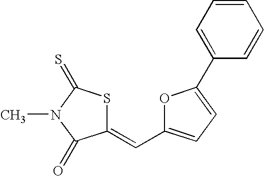 | (5Z)-3-methyl-5-[(5-phenyl-2-furyl)methylene]- thioxo-1,3-thiazolidin-4-one |
| 33 | 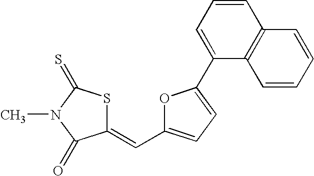 | (5Z)-3-methyl-5-{[5-(1-naphtthyl)-2- furyl]methylene}-2-thioxo-1,3-thiazolidin-4-one |
| 34 | 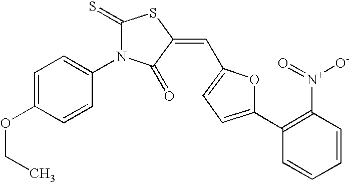 | (5E)-3-(4-ethoxyphenyl)-5-{[5-(2-nitrophenyl)-2- furyl]methylene}-2-thioxo-1,3-thiazolidin-4-one |
| 35 | 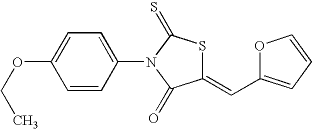 | (5Z)-3-(4-ethoxyphenyl)-5-(2-furylmethylene)-2- thioxo-1,3-thiazolidin-4-one |
| 36 | 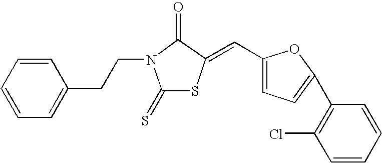 | (5Z)-5-{[5-(2-chlorophenyl)-2-furyl]methylene}-3- (2-phenylethyl)-2-thioxo-1,3-thiazolidin-4-one |
| 37 | 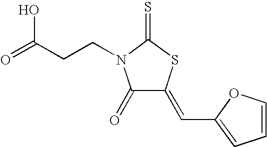 | 3-[(5Z)-5-(2-furylmethylene)-4-oxo-2-thioxo-1,3- thiazolidin-3-yl]propanoic acid |
| 38 | 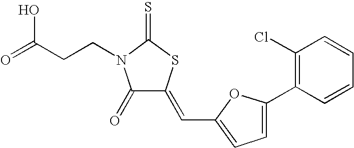 | 3-((5Z)-5-{[5-(2-chlorophenyl)-2-furyl]methylene}- 4-oxo-2-thioxo-1,3-thiazolidin-3-yl)propanoic acid |
| 39 | 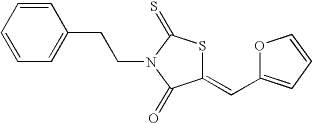 | (5Z)-5-(2-furylmethylene)-3-(2-phenylethyl)-2- thioxo-1,3-thiazolidin-4-one |
| 40 | 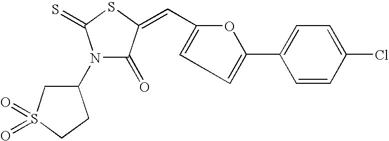 | (5E)-5-{[5-(4-chlorophenyl)-2-furyl]methylene}-3- (1,1-dioxidotetrahydro-3-thienyl)-2-thioxo-1,3- thiazolidin-4-one |
| 41 | 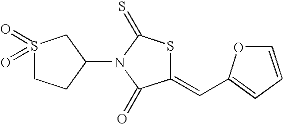 | (5Z)-3-(1,1-dioxidotetrahydro-3-thienyl)-5-(2- furylmethylene)-2-thioxo-1,3-thiazolidin-4-one |
| 42 | 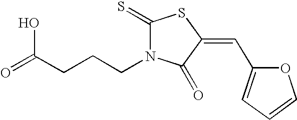 | 4-[(5E)-5-(2-furylmethylene)-4-oxo-2-thioxo-1,3- thiazolidin-3-yl]butanoic acid |
| 43 | 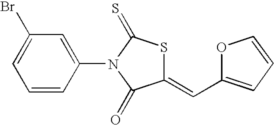 | (5Z)-3-(3-bromophenyl)-5-(2-furylmethylene)-2- thioxo-1,3-thiazolidin-4-one |
| 44 | 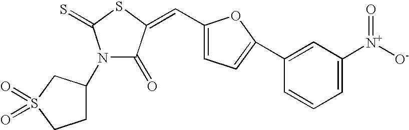 | (5E)-3-(1,1-dioxidotetrahydro-3-thienyl)-5-{[5-(3- nitrophenyl)-2-furyl]methylene}-2-thioxo-1,3- thiazolidin-4-one |
| 45 | 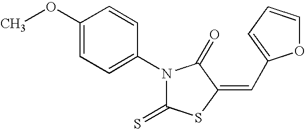 | (5E)-5-(2-furylmethylene)-3-(4-methoxyphenyl)-2- thioxo-1,3-thiazolidin-4-one |
| 46 | 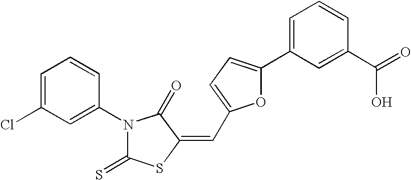 | 3-(5-{(E)-[3-(3-chlorophenyl)-4-oxo-2-thioxo-1,3- thiazolidin-5-ylidene]methyl}-2-furyl)benzoic acid |
| 47 | 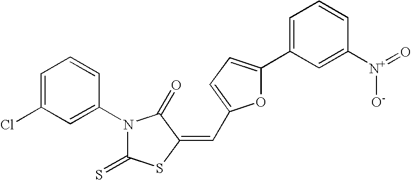 | (5E)-3-(3-chlorophenyl)-5-{[5-(3-nitrophenyl)-2- furyl]methylene}-2-thioxo-1,3-thiazolidin-4-one |
| 48 | 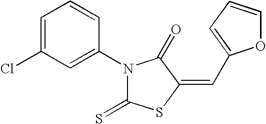 | (5E)-3-(3-chlorophenyl)-5-(2-furylmethylene)-2- thioxo-1,3-thiazolidin-4-one |
| 49 | 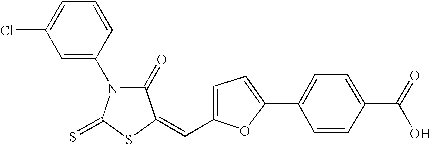 | 4-(5-[(E)-[3-(3-chlorophenyl)-4-oxo-2-thioxo-1,3- thiazolidin-5-ylidene]methyl}-2-furyl)benzoic acid |
| 50 | 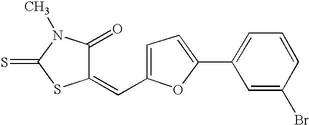 | (5E)-5-{[5-(3-bromophenyl)-2-furyl]methylene]-3- methyl-2-thioxo-1,3-thiazolidin-4-one |
| 51 | 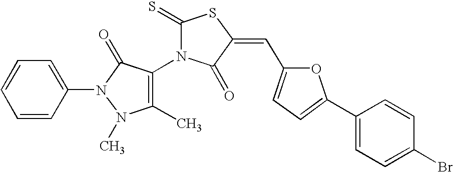 | (5E)-5-{[5-(4-bromophenyl)-2-furyl]methylene}-3- (1,5-dimethyl-3-oxo-2-phenyl-2,3-dihydro-1H- pyrazol-4-yl)-2-thioxo-1,3-thiazolidin-4-one |
| 52 | 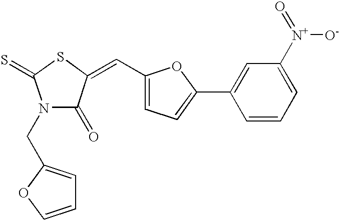 | (5E)-3-(2-furylmthyl)-5-{[5-(3-nitrophenyl)-2- furyl]methylene}-2-thioxo-1,3-thiazolidin-4-one |
| 53 | 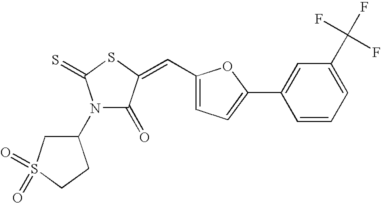 | (5E)-3-(1,1-dioxidotetrahydro-3-thienyl)-2-thioxo- 5-({5-[3-(trifluoromethyl)phenyl]-2- furyl}methylene)-1,3-thiazolidin-4-one |
| 54 | 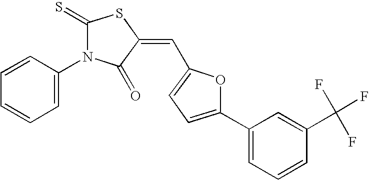 | (5E)-3-benzyl-2-thioxo-5-({5-[3- (trifluoromethyl)phenyl]-2-furyl}methylene)-1,3- thiazolidin-4-one |
| 55 | 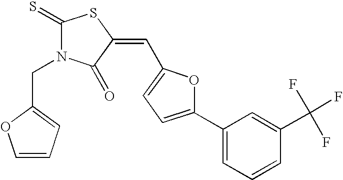 | (5E)-3-(2-furylmethyl)-2-thioxo-5-({5-[3- (trifluoromethyl)phenyl]-2-furyl}methylene)-1,3- thiazolidin-4-one |
| 56 | 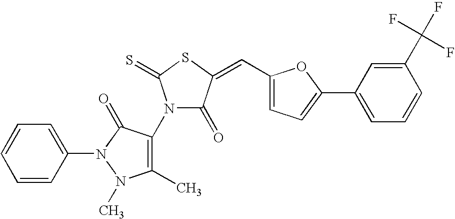 | (5E)-3-(1,5-dimethyl-3-oxo-2-phenyl-2,3-dihydro- 1H-pyrazol-4-yl)-2-thioxo-5-({5-[3- (trifluoromethyl)phenyl]-2-furyl}methylene)-1,3- thiazolidin-4-one |
| 57 | 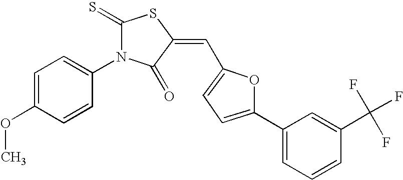 | (5E)-3-(4-methoxyphenyl)-2-thioxo-5-({5-[3- (trifluoromethyl)phenyl]-2-furyl}methylene)-1,3- thiazolidin-4-one |
| 58 | 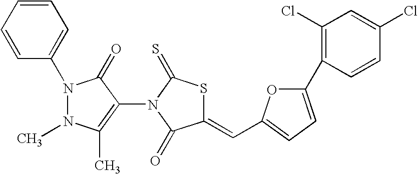 | (5Z)-5-{[5-(2,4-dichlorophenyl)-2-furyl]methylene}- 3-(1,5-dimethyl-3-oxo-2-phenyl-2,3-dihydro-1H- pyrazol-4-yl)-2-thioxo-1,3-thiazolidin-4-one |
| 59 | 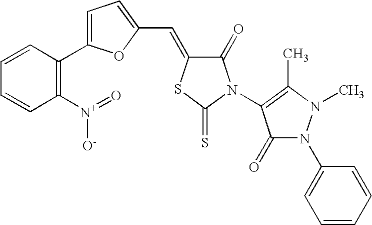 | (5Z)-3-(1,5-dimethyl-3-oxo-2-phenyl-2,3-dihydro- 1H-pyrazol-4-yl)-5-{[5-(2-nitrophhenyl)-2- furyl]methylene)-2-thioxo-1,3-thiazolidin-4-one |
| 60 | 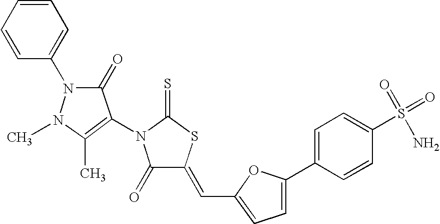 | 4-(5-{(Z)-[3-(1,5-dimethyl-3-oxo-2-phenyl-2,3- dihydro-1H-pyrazol-4-yl)-4-oxo-2-thioxo-1,3- thiazolidin-5-ylidene]methyl}-2- furyl)benzenesulfonamide |
| 61 | 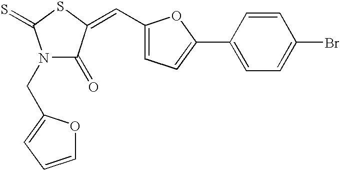 | (5E)-5-{[5-(4-bromophenyl)-2-furyl}methylene)-3- (2-furylmethyl)-2-thioxo-1,3-thiazolidin-4-one |
| 62 | 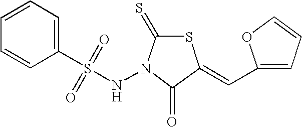 | N-[(5Z)-5-(2-furylmethylene)-4-oxo-2-thioxo-1,3- thiazolidin-3-yl]benzenesulfonamide |
| 63 | 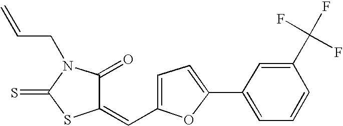 | (5E)-3-allyl-2-thioxo-5-({5-[3- (trifluoromethyl)phenyl]-2-furyl}methylene)-1,3- thiazolidin-4-one |
| 64 | 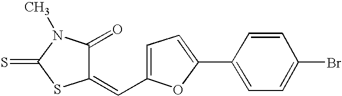 | (5E)-5-{[5-(4-bromophenyl)-2-furyl]methylene}-3- methyl-2-thioxo-1,3-thiazolidin-4-one |
| 65 | 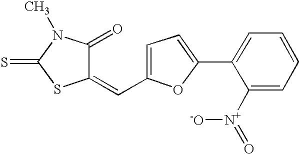 | (5E)-3-methyl-5-{[5-(2-nitrophenyl)-2- furyl]mthylene}-2-thioxo-1,3-thiazolidin-4-one |
| 66 | 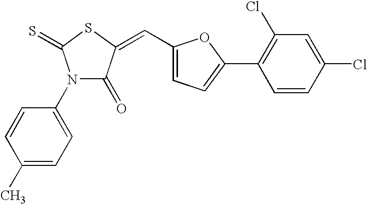 | (5E)-5-{[5-(2,4-dichlorophenyl)-2-furyl]methylene}- 3-(4-methylphenyl)-2-thioxo-1,3-thiazolidin-4-one |
| 67 | 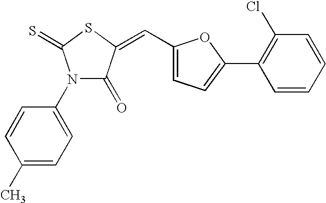 | (5E)-5-{[5-(2-chlorophenyl)-2-furyl]methylene}-3- (4-methylphenyl)-2-thioxo-1,3-thiazolidin-4-one |
| 68 | 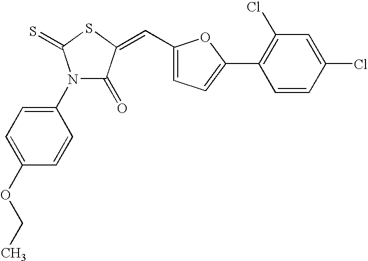 | (5E)-5-{[5-(2,4-dichlorophenyl)-2-furyl]methylene}- 3-(4-ethoxyphenyl)-2-thioxo-1,3-thiazolidin-4-one |
| 69 | 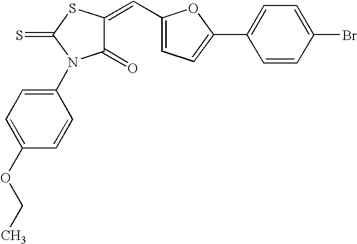 | (5E)-5-{[5-(4-bromophenyl)-2-furyl]mthylene}-3- (4-ethoxyphenyl)-2-thioxo-1,3-thiazolidin-4-one |
| 70 | 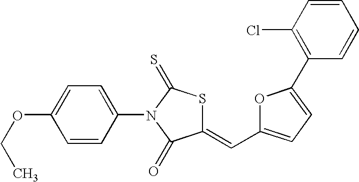 | (5Z)-5-{[5-(2-chlorophenyl)-2-furyl]methylene}-3- (4-ethoxyphenyl)-2-thioxo-1,3-thiazolidin-4-one |
| 71 | 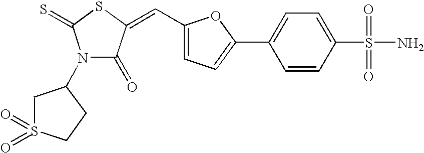 | 4-(5-{(E)-[3-(1,1-dioxidotetrahydro-3-thienyl)-4- oxo-2-thioxo-1,3-thiazolidin-5-ylidene]methyl}-2- furyl)benzenesulfonamide |
| 72 | 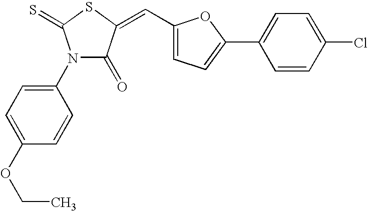 | (5E)-5-{[5-(4-chlorophenyl)-2-furyl]methylene}-3- (4-ethoxyphenyl)-2-thioxo-1,3-thiazolidin-4-one |
| 73 | 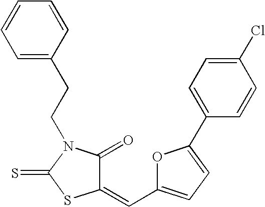 | (5E)-5-{[5-(4-chlorophenyl)-2-furyl]mthylene}-3- (2-phenylethyl)-2-thioxo-1,3-thiazolidin-4-one |
| 74 | 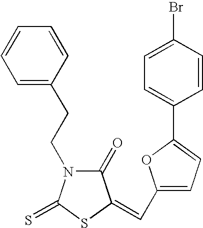 | (5E)-5-{[5-(4-bromophenyl)-2-furyl]methylene}-3- (2-phenylethyl)-2-thioxo-1,3-thiazoliddin-4-one |
| 75 | 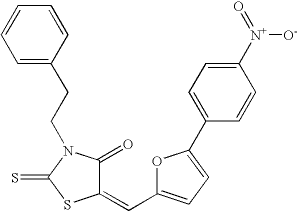 | (5E)-5-{[5-(4-nitrophenyl)-2-furyl]methylene}-3-(2- phenylethyl)-2-thioxo-1,3-thiazolidin-4-one |
| 76 | 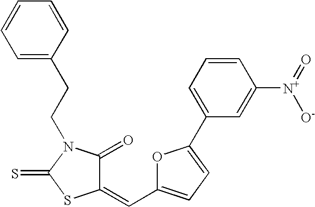 | (5E)-5-{[5-(3-nitrophenyl)-2-furyl]methylene}-3-(2- phenylethyl)-2-thioxo-1,3-thiazolidin-4-one |
| 77 | 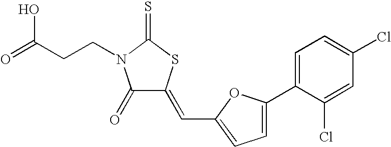 | 3-((5Z)-5-{[5-(2,4-dichlorophenyl)-2- furyl]methylene}-4-oxo-2-thioxo-1,3-thiazolidin-3- yl)propanoic acid |
| 78 | 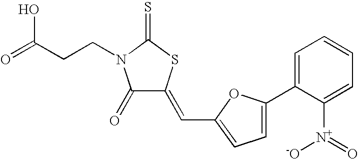 | 3-((5Z)-5-{[5-(2-nitrophenyl)-2-furyl]methylene}-4- oxo-2-thioxo-1,3-thiazolidin-3-yl)propanoic acid |
| 79 | 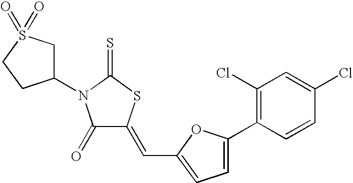 | (5Z)-5-{[5-(2,4-dichlorophenyl)-2-furyl]methylene}- 3-(1,1-dioxidotetrahydro-3-thienyl)-2-thioxo-1,3- thiazolidin-4-one |
| 80 | 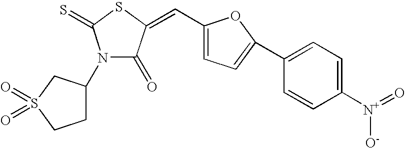 | (5E)-3-(1,1-dioxidotetrahydro-3-thienyl)-5-{[5-(4- nitrophenyl)-2-furyl]methylene}-2-thioxo-1,3- thiazolidin-4-one |
| 81 | 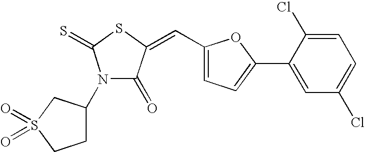 | (5E)-5-{[5-(2,5-dichlorophenyl)-2-furyl]mthylene}- 3-(1,1-dioxidotetrahydro-3-thienyl)-2-thioxo-1,3- thiazolidin-4-one |
| 82 | 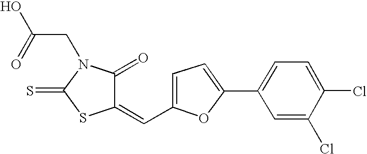 | ((5E)-5-{[5-(3,4-dichlorophenyl)-2- furyl]methylene}-4-oxo-2-thioxo-1,3-thiazolidin-3- yl)acetic acid |
| 83 | 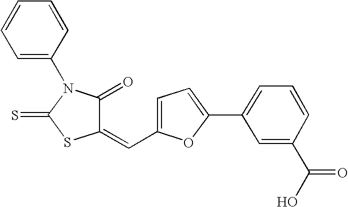 | 3-{5-[(E)-(4-oxo-3-phenyl-2-thioxo-1,3-thiazolidin-5- ylidene)methyl]-2-furyl}benzoic acid |
| 84 | 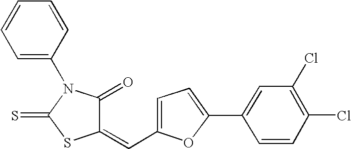 | (5E)-5-{[5-(3,4-dichlorophenyl)-2-furyl]methylene}- 3-phenyl-2-thioxo-1,3-thiazolidin-4-one |
| 85 | 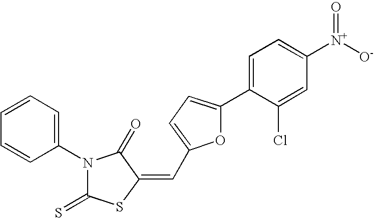 | (5E)-5-{[5-(2-chloro-4-nitrophenyl)-2- furyl]methylene}-3-phenyl-2-thioxo-1,3-thiazolidin- 4-one |
| 86 | 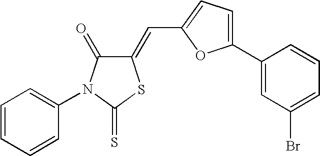 | (5Z)-5-{[5-(3-bromophenyl)-2-furyl]methylene}-3- phenyl-2-thioxo-1,3-thiazolidin-4-one |
| 87 | 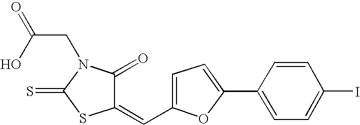 | ((5E)-5-{[5-(4-iodophenyl)-2-furyl]methylene}-4- oxo-2-thioxo-1,3-thiazolidin-3-yl)acetic acid |
| 88 | 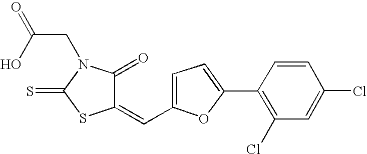 | ((5E)-5-{[5-(2,4-dichlorophenyl)-2- furyl]methylene}-4-oxo-2-thioxo-1,3-thiazolidin-3- yl)acetic acid |
| 89 | 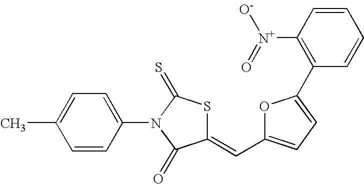 | (5Z)-3-(4-methylphenyl)-5-{[5-(2-nitrophenyl)-2- furyl]methylene}-2-thioxo-1,3-thiazolidin-4-one |
| 90 | 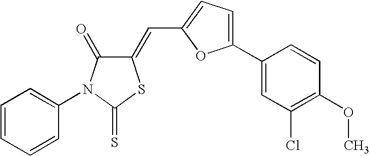 | (5Z)-5-{[5-(3-chloro-4-methoxyphenyl)-2- furyl]methylene}-3-phenyl-2-thioxo-1,3-thiazolidin- 4-one |
| 91 | 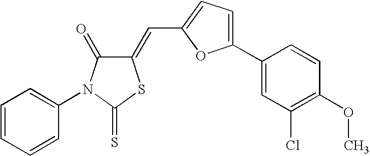 | (5Z)-5-{[5-(3-chloro-4-methoxyphenyl)-2- furyl]methylene}-3-phenyl-2-thioxo-1,3-thiazolidin- 4-one |
| 92 | 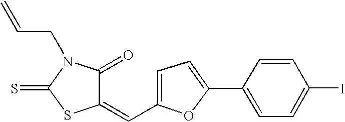 | (5E)-3-allyl-5-{[5-(4-iodophenyl)-2-furyl]methylene}- 2-thioxo-1,3-thiazolidin-4-one |
| 93 | 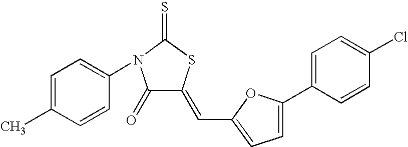 | (5Z)-5-{[5-(4-chlorophenyl)-2-furyl]methylene}-3- (4-methylphenyl)-2-thioxo-1,3-thiazolidin-4-one |
| 94 | 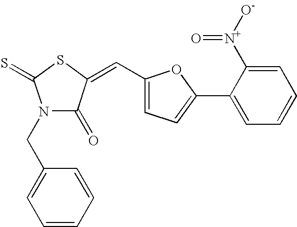 | (5E)-3-benzyl-5-{[5-(2-nitrophenyl)-2- furyl]methylene}-2-thioxo-11,3-thiazolidin-4-one |
| 95 | 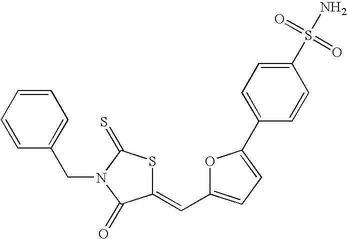 | 4-{5-[(Z)-(3-benzyl-4-oxo-2-thioxo-1,3-thiazolidin-5- ylidene)methyl]-2-furyl}benzenesulfonamide |
| 96 | 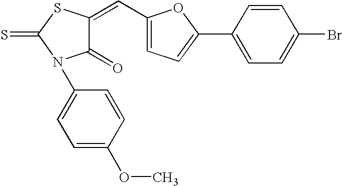 | (5E)-5-{[5-(4-bromophenyl)-2-furyl]methylene}-3- (4-methoxyphenyl)-2-thioxo-1,3-thiazolidin-4-one |
| 97 | 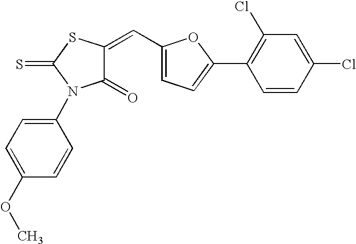 | (5E)-5-{[5-(2,4-dichlorophenyl)-2-furyl]methylene}- 3-(4-methoxyphenyl)-2-thioxo-1,3-thiazolidin-4- one |
| 98 | 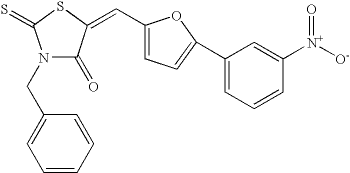 | (5E)-3-benzyl-5-{[5-(3-nitrophenyl)-2- furyl]methylene}-2-thioxo-1,3-thiazolidin-4-one |
| 99 | 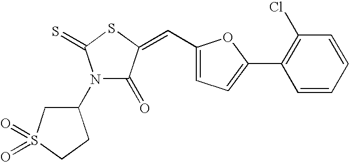 | (5E)-5-{[5-(2-chlorophenyl)-2-furyl]methylene}-3- (1,1-dioxidotetrahydro-3-thienyl)-2-thioxo-1,3- thiazolidin-4-one |
| 100 | 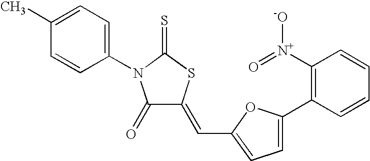 | (5Z)-3-(4-methylphenyl)-5-{[5-(2-nitrophenyl)-2- furyl]methylene}-2-thioxo-1,3-thiazolidin-4-one |
| 101 | 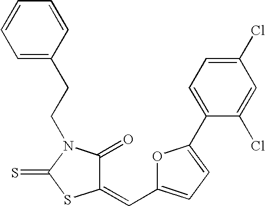 | (5E)-5-{[5-(2,4-dichlorophenyl)-2-furyl]methylene}- 3-(2-phenylethyl)-2-thioxo-1,3-thiazolidin-4-one |
| 102 | 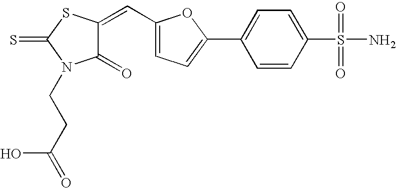 | 3-[(5E)-5-({5-[4-(aminosulfonyl)phenyl]-2- furyl}methylene)-4-oxo-2-thioxo-1,3-thiazolidin-3- yl]propanoic acid |
| 103 | 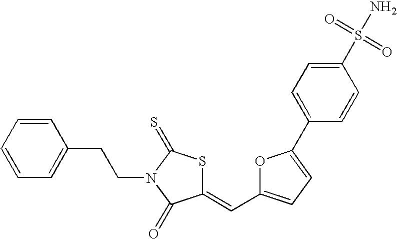 | 4-(5-{(Z)-[4-oxo-3-(2-phenylethyl)-2-thioxo-1,3- thiazolidin-5-ylidene]methyl}-2- furyl)benzenesulfonamide |
| 104 | 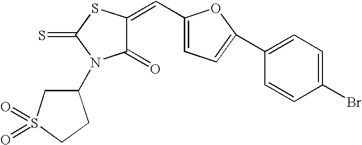 | (5E)-5-{[5-(4-bromophenyl)-2-furyl]methylene}-3- (1,1-dioxidotetrahydro-3-thienyl)-2-thioxo-1,3- thiazolidin-4-one |
| 105 | 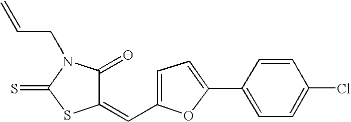 | (5E)-3-allyl-5-{[5-(4-chlorophenyl)-2- furyl]methylene}-2-thioxo-1,3-thiazolidin-4-one |
| 106 | 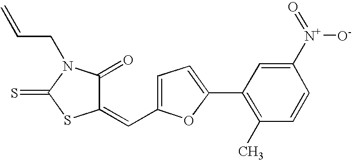 | (5E)-3-allyl-5-{[5-(2-methyl-5-nitrophenyl)-2- furyl]methylene}-2-thioxo-1,3-thiazolidin-4-one |
| 107 | 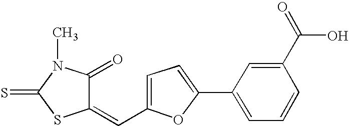 | 3-{5-[(E)-(3-methyl-4-oxo-2-thioxo-1,3-thiazolidin- 5-ylidene)methyl]-2-furyl}benzoic acid |
| 108 | 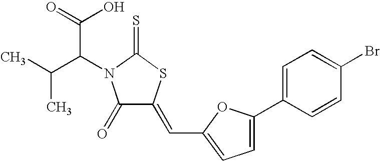 | 2-((5Z)-5-{[5-(4-bromophenyl)-2-furyl]methylene}- 4-oxo-2-thioxo-1,3-thiazolidin-3-yl)-3- methylbutyric acid |
| 109 | 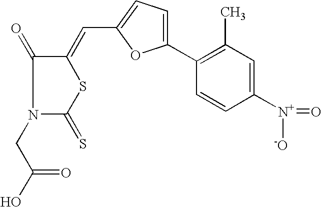 | ((5Z)-5-{[5-(2-methyl-4-nitrophenyl)-2- furyl]methylene}-4-oxo-2-thioxo-1,3-thiazolidin-3- yl)acetic acid |
| 110 | 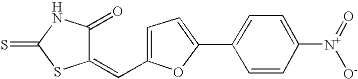 | (5E)-5-{[5-(4-nitrophenyl)-2-furyl]methylene}-2- thioxo-1,3-thiazolidin-4-one |
| 111 | 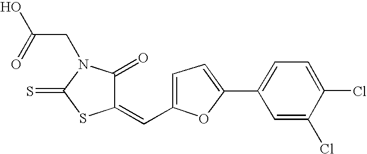 | ((5E)-5-{[5-(3,4-dichlorophenyl)-2- furyl]methylene}-4-oxo-2-thioxo-1,3-thiazolidin-3- yl)acetic acid |
| 112 | 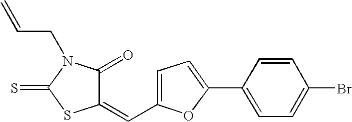 | (5E)-3-allyl-5-{[5-(4-bromophenyl)-2- furyl]methylene}-2-thioxo-1,3-thiazolidin-4-one |
| 113 | 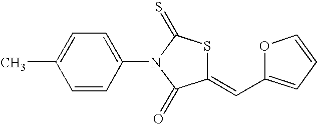 | (5Z)-5-(2-furylmethylene)-3-(4-methylphenyl)-2- thioxo-1,3-thiazolidin-4-one |
| 114 | 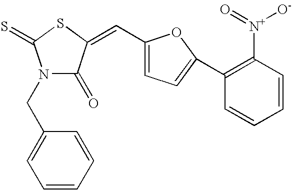 | (5E)-3-benzyl-5-{[5-(2-nitrophenyl)-2- furyl]methylene}-2-thioxo-1,3-thiazolidin-4-one |
| 115 | 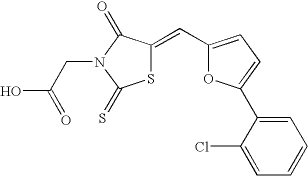 | ((5Z)-5-{[5-(2-chlorophenyl)-2-furyl]methylene}-4- oxo-2-thioxo-1,3-thiazolidin-3-yl)acetic acid |
| 116 | 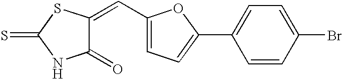 | (5E)-5-{[5-(2-bromophenyl)-2-furyl]methylene}-2- thioxo-1,3-thiazolidin-4-one |
| 117 | 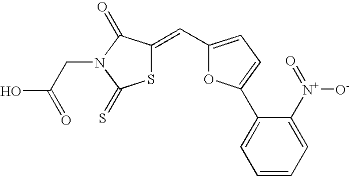 | ((5Z)-5-{[5-(2-nitrophenyl)-2-furyl]methylene}-4- oxo-2-thioxo-1,3-thiazolidin-3-yl)acetic acid |
| 118 | 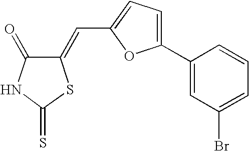 | (5Z)-5-{[5-(3-bromophenyl)-2-furyl]methylene}-2- thioxo-1,3-thiazolidin-4-one |
| 119 | 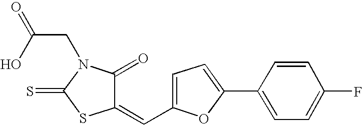 | ((5E)-5-{[5-(4-fluorophenyl)-2-furyl]methylene}-4- oxo-2-thioxo-1,3-thiazolidin-3-yl)acetic acid |
| 120 | 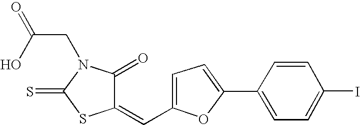 | ((5E)-5-{[5-(4-iodophenyl)-2-furyl]methylene}-4- oxo-2-thioxo-1,3-thiazolidin-3-yl)acetic acid |
| 121 | 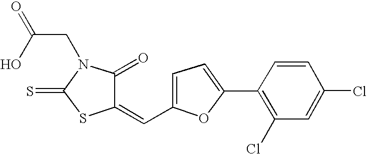 | ((5E)-5-{[5-(2,4-dichlorophenyl)-2- furyl]methylene}-4-oxo-2-thioxo-1,3-thiazolidin-3- yl)acetic acid |
| 122 | 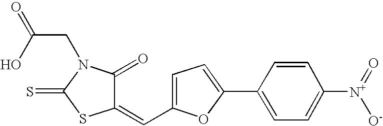 | ((5E)-5-{[5-(4-nitrophenyl)-2-furyl]methylene}-4- oxo-2-thioxo-1,3-thiazolidin-3-yl)acetic acid |
| 123 | 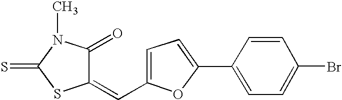 | (5E)-5-{[5-(4-bromophenyl)-2-furyl]methylene}-3- methyl-2-thioxo-1,3-thiazolidin-4-one |
| 124 | 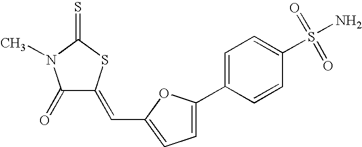 | 4-{5-[(Z)-(3-methyl-4-oxo-2-thioxo-1,3-thiazolidin-5- ylidene)methyl]-2-furyl}benzenesulfonamide |
| 125 | 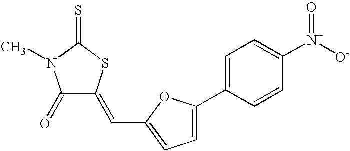 | (5Z)-3-methyl-5-{[5-(4-nitrophenyl)-2- furyl]methylene}-2-thioxo-1,3-thiazolidin-4-one |
| 126 | 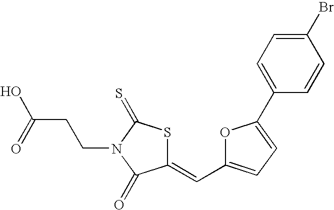 | 3-((5Z)-5-{[5-(4-bromophenyl)-2-furyl]methylene}- 4-oxo-2-thioxo-1,3-thiazolidin-3-yl)propanoic acid |
| 127 | 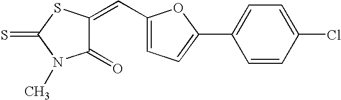 | (5E)-5-{[5-(4-chlorophenyl)-2-furyl]methylene}-3- methyl-2-thioxo-1,3-thiazolidin-4-one |
| 128 | 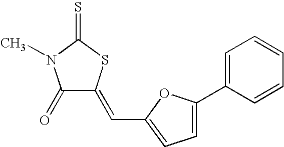 | (5Z)-33-methyl-5-[(5-phenyl-2-furyl)methylene]-2- thioxo-1,3-thiazolidin-4-one |
| 129 | 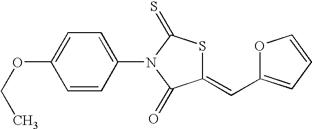 | (5Z)-3-(4-ethoxyphenyl)-5-(2-furylmethylene)-2- thioxo-1,3-thiazolidin-4-one |
| 130 | 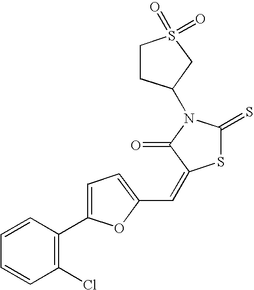 | (5E)-5-{[5-(2-chlorophenyl)-2-furyl]methylene}-3- (1,1-dioxidotetrahydro-3-thienyl)-2-thioxo-1,3- thiazolidin-4-one |
| 131 | 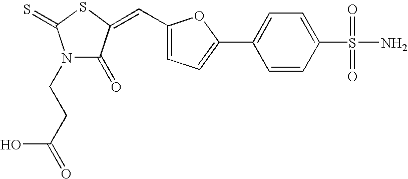 | 3-[(5E)-5-({5-[4-(aminosulfonyl)phenyl]-2- furyl}methylene)-4-oxo-2-thioxo-1,3-thiazolidin-3- yl]propanoic acid |
| 132 | 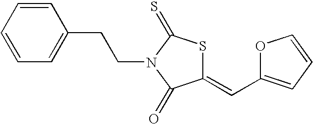 | (5Z)-5-(2-furylmethylene)-3-(2-phenylethyl)-2- thioxo-1,3-thiazolidin-4-one |
| 133 | 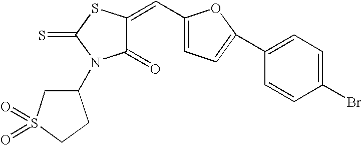 | (5E)-5-{[5-(4-bromophenyl)-2-furyl]methylene-56 -3- (1,1-dioxidotetrahydro-3-thienyl)-2-thioxo-1,3- thiazolidin-4-one |
| 134 | 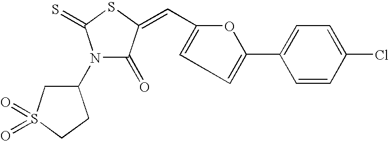 | (5E)-5-{[5-(4-chlorophenyl)-2-furyl]methylene}-3- (1,1-dioxidotetrahydro-3-thienyl)-2-thioxo-1,3- thiazolidin-4-one |
| 135 | 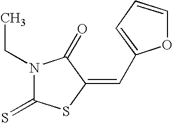 | (5E)-3-ethyl-5-(2-furylmethylene)-2-thioxo-1,3- thiazolidin-4-one |
| 136 | 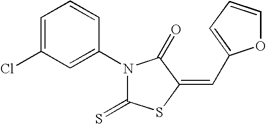 | (5E)-3-(3-chlorophenyl)-5-(2-furylmethylene)-2- thioxo-1,3-thiazolidin-4-one |
| 137 | 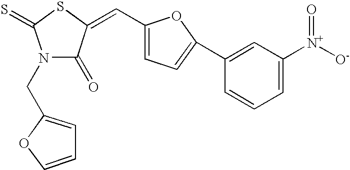 | (5E)-3-(2-furylmethyl)-5-{[5-(3-nitrophenyl)-2- furyl]methylene}-2-thioxo-1,3-thiazolidin-4-one |
| 138 | 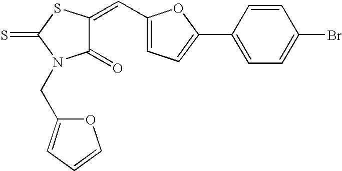 | (5E)-5-{[5-(4-bromophenyl)-2-furyl]methylene}-3- (2-furylmethyl)-2-thioxo-1,3-thiazolidin-4-one |
| 139 | 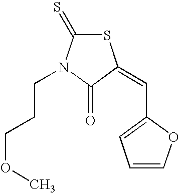 | (5E)-5-(2-furylmethylene)-3-(3-methoxypropyl)-2- thioxo-1,3-thiazolidin-4-one |
| 140 | 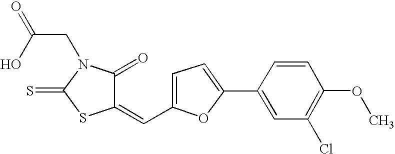 | ((5E)-5-{[5-(3-chloro-4-methoxyphenyl)-2- furyl]methylene}-4-oxo-2-thioxxo-1,3-thiazolidin-3- yl)acetic acid |
| 141 | 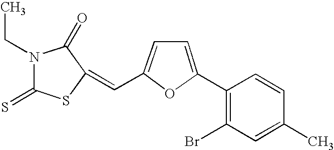 | (5E)-5-{[5-(2-bromo-4-methylphenyl)-2- furyl]mthylene}-2-thioxo-1,3-thiazolidin-4-one |
| 142 | 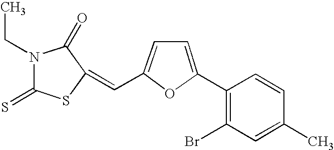 | (5E)-55-{[5-(2-bromo-4-methylphenyl)-2- furyl]methylene}-3-ethyl-2-thioxo-1,3-thiazolidin-4- one |
| 143 | 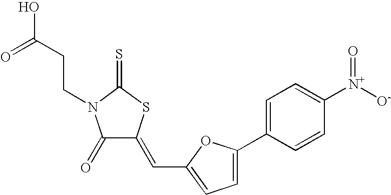 | 3-((5Z)-5-{[5-(4-nitrophenyl)-2-furyl]methylene}-4- oxo-2-thioxo-1,3-thiazolidin-3-yl)propanoic acid |
| 144 | 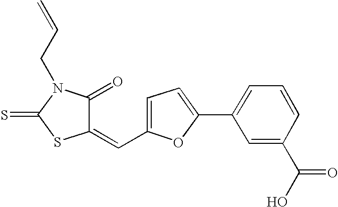 | 3-[5-[(E)-(3-allyl-4-oxo-2-thioxo-1,3-thiazolidin-5- ylidene)methyl]-2-furyl}benzoic acid |
| 145 | 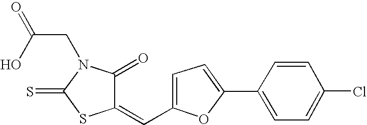 | ((5E)-5-{[5-(4-chlorophenyl)-2-furyl]methylene}-4- oxo-2-thioxo-1,3-thiazolidin-3-yl)acetic acid |
| 146 | 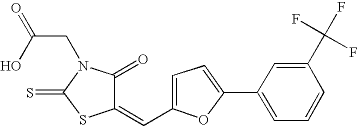 | [(5E)-4-oxo-2-thioxo-5-({5-[3- (trifluoromethyl)phenyl]-2-furyl]methylene)-1,3- thiazolidin-3-yl]acetic acid |
| 147 | 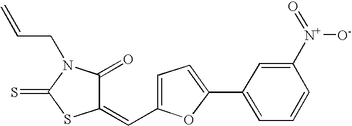 | (5E)-3-allyl-5-{[5-(3-nitrophenyl)-2- furyl]methylene}-2-thioxo-1,3-thiazolidin-4-one |
| 148 | 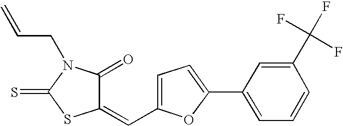 | (5E)-3-allyl-2-thioxo-5-({5-[3- (trifluoromethyl)phenyl]-2-furyl}methylene)-1,3- thiazolidin-4-one |
| 149 | 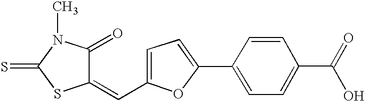 | 4-{5-[(E)-(3-methyl-4-oxo-2-thioxxo-1,3-thiazolidin- 5-ylidene)methyl]-2-furyl}benzoic acid |
| 150 | 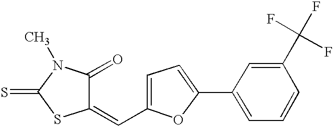 | (5E)-3-methyl-2-thioxo-5-({5-[3- (trifluoromethyl)phenyl]-2-furyl}methylene}-1,3- thiazolidin-4-one |
| 151 | 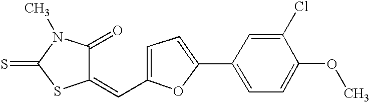 | (5E)-5-{[5-(3-chlorro-4-methoxyphenyl)-2- furyl]methylene}-3-methyl-2-thioxo-1,3-thiazolidin- 4-one |
| 152 | 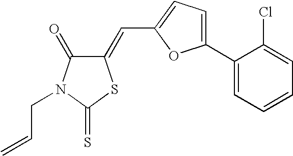 | (5Z)-3-allyl-5-{[5-(2-chlorophhenyl)-2- furyl]methylene}-2-thioxo-1,3-thiazolidin-4-one |
| 153 | 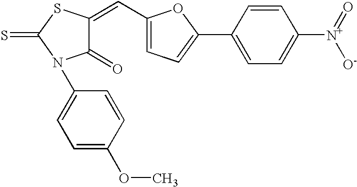 | (5E)-3-(4-methoxyphenyl)-5-{[5-(4-nitrophenyl)-2- furyl]methylene}-2-thioxo-1,3-thiazolidin-4-one |
| 154 | 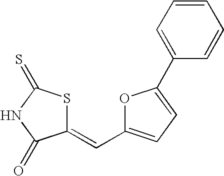 | (5Z)-5-[(5-phenyl-2-furyl)methylene]-2-thioxo-1,3- thiazolidin-4-one |
| 155 | 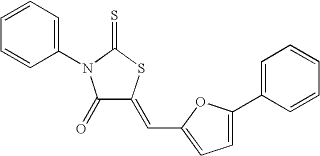 | (5Z)-3-phenyl-5-[(5-phenyl-2-furyl)methylene]-2- thioxo-1,3-thiazolidin-4-one |
| 156 | 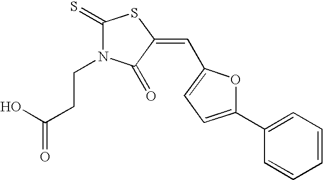 | 3-{(5E)-4-oxo-5-[(5-phenyl-2-furyl)methylene]-2- thioxo-1,3-thiazolidin-33-yl}propanoic acid |
| 157 | 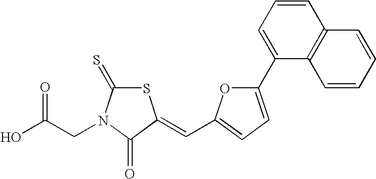 | ((5Z)-5-{[5-(1-naphthyl)-2-furyl]methylene}-4-oxo-2- thioxo-1,3-thiazolidin-3-yl)acetic acid |
| 158 | 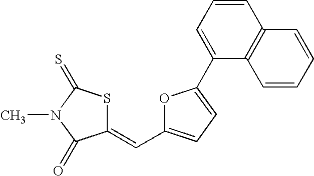 | (5Z)-3-methyl-55-{[5-(1-naphthyl)-2- furyl]methylene}-2-thioxo-1,3-thiazolidin-4-one |
| 159 | 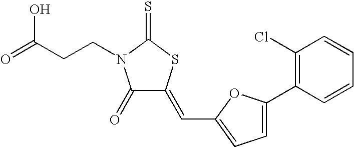 | 3-((5Z)-5-{[5-(2,4-dichlorophenyl)-2-furyl]methylene}- 4-oxo-2-thioxo-1,3-thiazolidin-3-yl)propanoic acid |
| 160 | 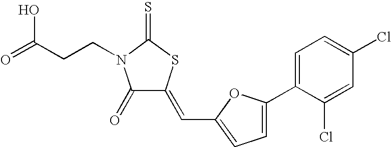 | 3-((5Z)-5-{[5-(2,4-dichlorophenyl)-2- furyl]methylene}-4-oxo-2-thioxo-1,3-thiazolidin-3- yl)propanoic acid |
| 161 | 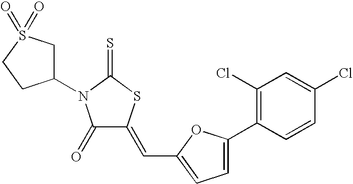 | (5Z)-5-{[5-(2,4-dichlorophenyl)-2-furyl]methylene}- 3-(1,1-dioxidotetrahydro-3-thienyl)-2-thioxo-1,3- thiazolidin-4-one |
| 162 | 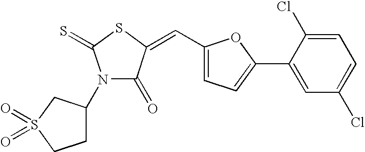 | (5E)-5-{[5-(2,5-dichlorophenyl)-2-furyl]methylene}- 3-(1,1-dioxidotetrahydro-3-thienyl)-2-thioxo-1,3- thiazolidin-4-one |
| 163 | 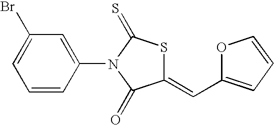 | (5Z)-3-(3-bromophenyl)-55-(2-furylmethylene)-2- thioxo-1,3-thiazolidin-4-one |
| 164 | 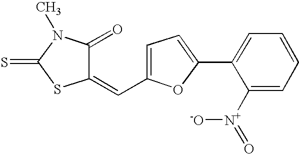 | (5E)-3-mthyl-5-{[5-(2-nitrophenyl)-2- furyl]methylene}-2-thioxo-1,3-thiazolidin-4-one |
| 165 | 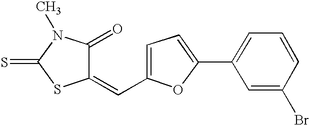 | (5E)-4-{[5-(3-bromophenyl)-2-furyl]methylene}-3- methyl-2-thioxo-1,3-thiazolidin-4-one |
| 166 | 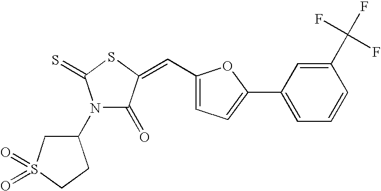 | (5E)-3-(1,1-dioxidotetrahydro-3-thienyl)-2-thioxo- 5-({5-[3-(trifluoromethyl)phenyl]-2- furyl}methylene)-1,3-thiazolidin-4-one |
| 167 | 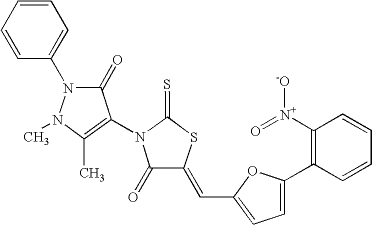 | (5Z)-3-(1,5-dimethyl-3-oxo-2-phenyl-2,3-dihydro- 1H-pyrazol-4-yl)-5-{[5-(2-nitropenyl)-2- furyl]methylene}-2-thioxo-1,3-thiazolidin-4-one |
| 168 | 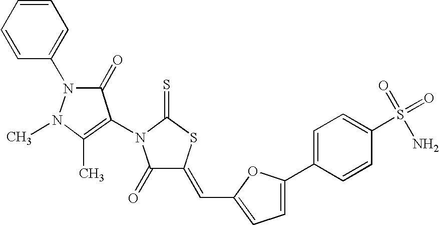 | 4-(5-{(Z)-[3-(1,5-dimethyl-3-oxo-2-phenyl-2,3- dihydro-1H-pyrazol-4-yl)-4-oxo-2-thioxo-1,3- thiazolidin-5-ylidene]methyl}-2- furyl)benzenesulfonamide |
| 169 | 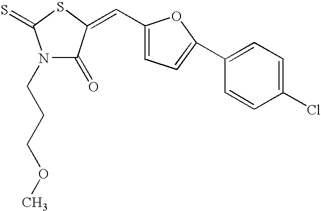 | (5E)-5-{[5-(4-chlorophenyl)-2-furyl]methylene}-3- (3-methoxypropyl)-2-thioxo-1,3-thiazolidin-4-one |
| 170 | 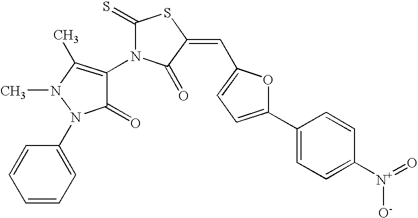 | (5E)-3-(1,5-dimthyl-3-oxo-2-phenyl-2,3-dihydro- 1H-pyrazol-4-yl)-5-{[5-(4-nitrophenyl)-2- furyl]methylene}-2-thioxo-1,3-thiazolidin-4-one |
| 171 | 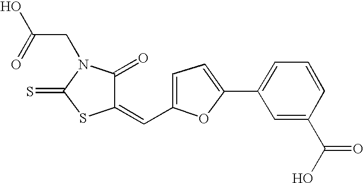 | 3-(5-[(E)-[3-(carboxymethyl)-4-oxo-2-thioxo-1,3- thiazolidin-5-ylidene]mthyl}-2-furyl)benzoic acid |
| 172 | 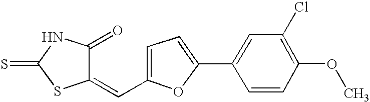 | (5E)-5-{[5-(3-chloro-4-methoxyphenyl)-2- furyl]methylene}-2-thioxo-1,3-thiazolidin-4-one |
| 173 | 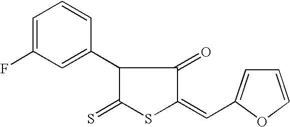 | (5E)-3-(3-fluorophenyl)-5-(2-furylmethylene)-2- thioxo-1,3-thiazolidin-4-one |
| 174 | 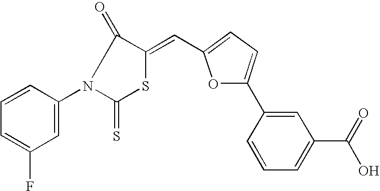 | 3-(5-{(Z)-[3-(3-fluorophenyl)-4-oxo-2-thioxo-1,3- thiazolidin-5-ylidene]methyl}-2-furyl)benzoic acid |
| 175 | 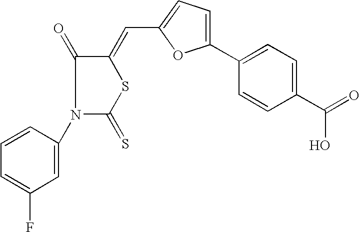 | 4-(5-{(Z)-[3-(3-fluorophenyl)-4-oxo-2-thioxo-1,3- thiazolidin-5-ylidene]methyl}-2-furyl)benzoic acid |
| 176 | 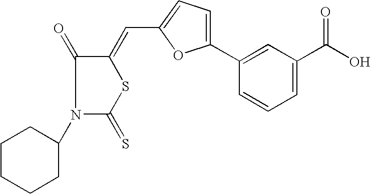 | 3-{5-[(Z)-(3-cyclohexyl-4-oxo-2-thioxo-1,3- thiazolidin-5-ylidene)methyl]-2-furyl}benzoic acid |
| 177 | 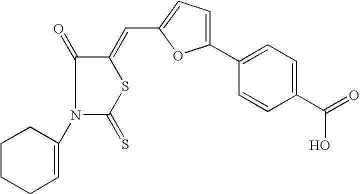 | 4-{5-[(Z)-(3-cyclohexyl-4-oxo-2-thioxo-1,3- thiazolidin-5-ylidene)methyl]-2-furyl}benzoic acid |
| 178 | 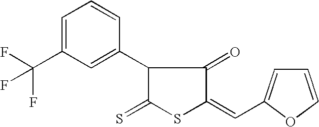 | (5E)-5-(2-furylmethylene)-2-thioxo-3-[3- (trifluoromethyl)phenyl]-1,3-thiazolidin-4-one |
| 179 | 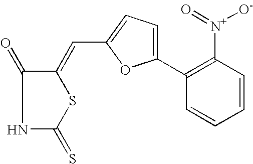 | (5Z)-5-{[5-(2-nitrophenyl)-2-furyl]methylene}-2- thioxo-1,3-thiazolidin-4-one |
| 180 | 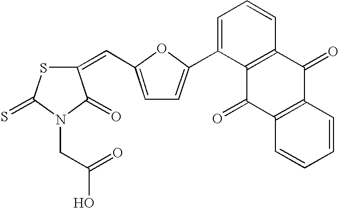 | ((5E)-5-{[5-(9,10-dioxo-9,10-dihydroanthracen-1- yl)-2-furyl]methylene}-4-oxo-2-thioxo-1,3- thiazolidin-3-yl)acetic acid |
| 181 | 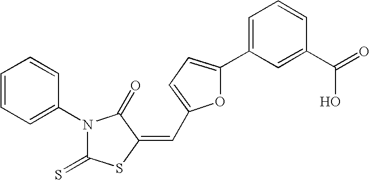 | 3-{5-[(E)-(4-oxo-3-phenyl-2-thioxo-1,3-thiazolidin-5- ylidene)methyl]-2-furyl}benzoic acid |
| 182 | 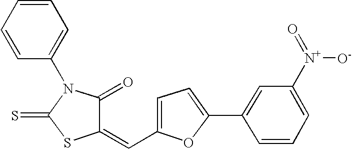 | (5E)-5-{[5-(3-nitrophenyl)-2-furyl]methylene}-3- phenyl-2-thioxo-1,3-thiazolidin-4-one |
| 183 | 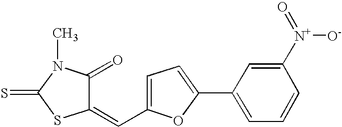 | (5E)-3-methyl-5-{[5-(3-nitrophenyl)-2- furyl]methylene}-2-thioxo-1,3-thiazolidin-4-one |
| 184 | 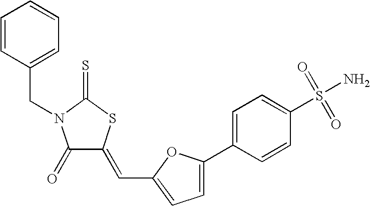 | 4-{5-[(Z)-(3-benzyl-4-oxo-2-thioxo-1,3-thiazolidin-5- ylidene)methyl]-2-furyl}benzenesulfonamide |
| 185 | 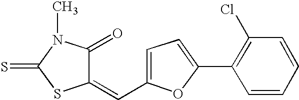 | (5E)-5-{[5-(2-chlorophenyl)-2-furylmethylene}-3- mthyl-2-thioxo-1,3-thiazolidin-4-one |
| 186 | 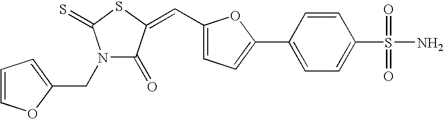 | 4-(5-{(E)-[3-(2-furylmethyl)-4-oxo-2-thioxo-1,3- thiazolidin-5-ylidene]methyl}-2- furyl)benzenesulfonamide |
| 187 | 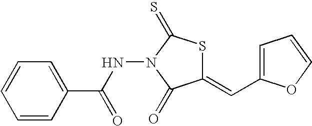 | N-[(5Z)-5-(2-furylmethylene)-4-oxo-2-thioxo-1,3- thiazolidin-3-yl]benzamide |
| 188 | 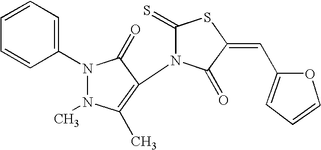 | (5E)-3-(1,5-dimethyl-3-oxo-2-phenyl-2,3-dihydro- 1H-pyrazol-4-yl)-5-(2-furylmethylene)-2-thioxo-1,3- thiazolidin-4-one |
| 189 | 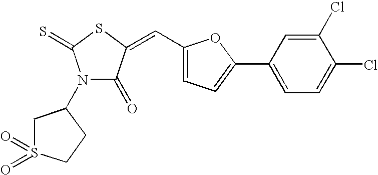 | (5E)-5-{[5-(3,4-dichlorophenyl)-2-furyl]methylene}- 3-(1,1-dioxidotetrahydro-3-thienyl)-2-thioxo-1,3- thiazolidin-4-one |
| 190 | 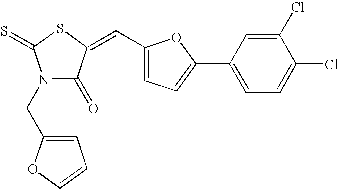 | (5E)-5-{[5-(3,4-dichlorophenyl)-2-furyl]methylene}- 3-(2-furylmethyl)-2-thioxo-1,3-thiazolidin-4-one |
| 191 | 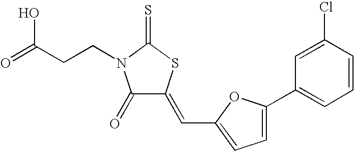 | 3-((5Z)-5-{[5-(3-chlorophenyl)-2-furyl]methylene}- 4-oxo-2-thioxo-1,3-thiazolidin-3-yl}propanoic acid |
| 192 | 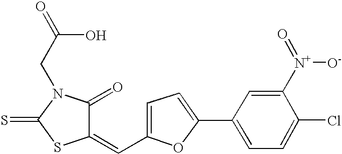 | ((5E)-5-{[5-(4-chloro-3-nitrophenyl)-2- furyl]methylene}-4-oxo-2-thioxo-1,3-thiazolidin-3- yl)acetic acid |
| 193 | 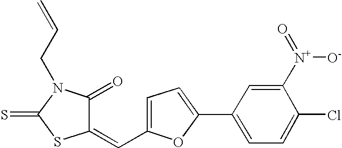 | (5E)-5-{[5-(4-chloro-3-nitrophenyl)-2- furyl]methylene}-3-propyl-2-thioxo-1,3-thiazolidin- 4-one |
| 194 | 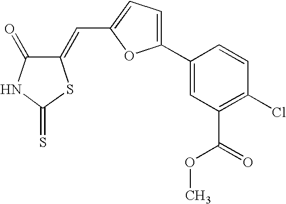 | methyl-2-chloro-5-{5-[(Z)-(4-oxo-2-thioxo-1,3- thiazolidin-5-ylidene)methyl]-2-furyl}benzoate |
| 195 | 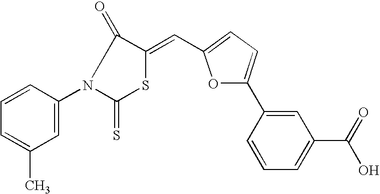 | 3-(5-{(Z)-[3-(3-methylphenyl)-4-oxo-2-thioxo-1,3- thiazolidin-5-ylidene]methyl}-2-furyl)benzoic acid |
| 196 | 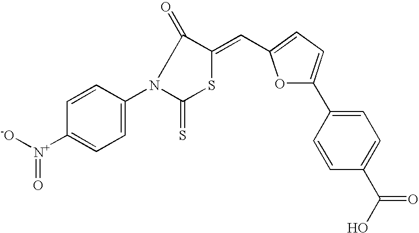 | 4-(5-{(Z)-[3-(4-nitrophenyl)-4-oxo-2-thioxo-1,3- thiazolidin-5-ylidene]methyl}-2-furyl)benzoic acid |
| 197 | 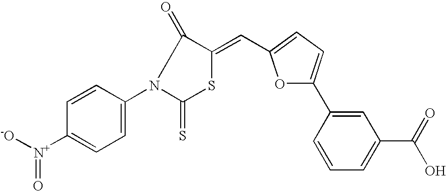 | 3-(5-{(Z)-[3-(4-nitrophenyl)-4-oxo-2-thioxo-1,3- thiazolidin-5-ylidene]methyl}-2-furyl)benzoic acid |
| 198 | 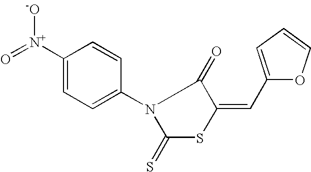 | (5E)-5-(2-furylmethylene)-3-(4-nitrophenyl)-2- thioxo-1,3-thiazolidin-4-one |
| 199 | 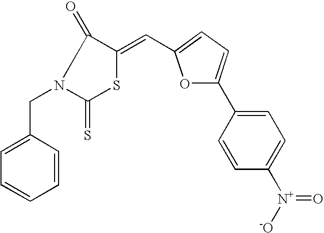 | (5Z)-3-benzyl-5-{[5-(4-nitrophenyl)-2- furyl]methylene}-2-thioxo-1,3-thiazolidin-4-one |
| 200 | 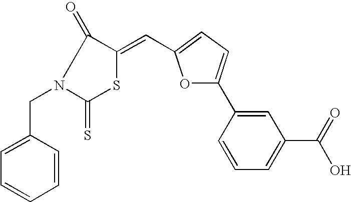 | 3-{5-[(Z)-(3-benzyl-4-oxo-2-thioxo-1,3-thiazolidin-5- ylidene)methyl]-2-furyl}benzoic acid |
| 201 | 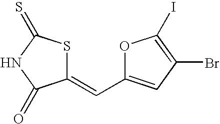 | (5Z)-5-[(4-bromo-5-iodo-2-furyl)methylene]-2- thioxo-1,3-thiazolidin-4-one |
| 202 | 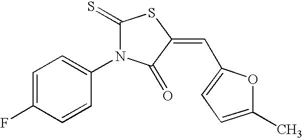 | (5E)-3-(4-fluorophenyl)-5-[(5-methyl-2- furyl)methylene]-2-thioxo-1,3-thiazolidin-4-one |
| 203 | 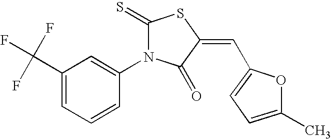 | (5E)-5-[(5-methyl-2-furyl)methylene]-2-thioxo-3-[3- (trifluoromethyl)phenyl]-1,3-thiazolidin-4-one |
| 204 | 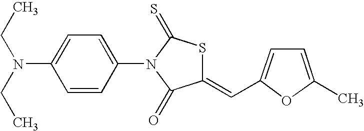 | (5Z)-3-[4-(diethylamino)phenyl]-5-[(5-methyl-2- furyl)methylene]-2-thioxo-1,3-thiazolidin-4-one |
| 205 | 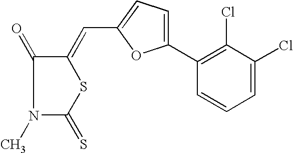 | (5Z)-5-{[5-(2,3-dichlorophenyl)-2-furyl]methylene}- 3-methyl-2-thioxo-1,3-thiazolidin-4-one |
| 206 | 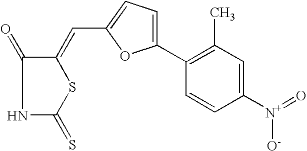 | (5Z)-5-{[5-(2-methyl-4-nitrophenyl)-2- furyl]mthylene}-2-thioxo-1,3-thiazolidin-4-one |
| 207 | 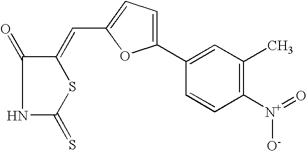 | (5Z)-5-{[5-(3-methyl-4-nitrophenyl)-2- furyl]methylene}-2-thioxo-1,3-thiazolidin-4-one |
| 208 | 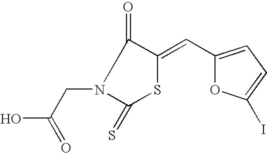 | {(5Z)-5-[(5-iodo-2-furyl)methylene]-4-oxo-2-thioxo- 1,3-thiazolidin-3-yl}acetic acid |
| 209 | 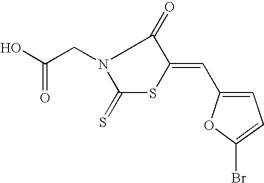 | {(5Z)-5-[(5-bromo-2-furyl)methylene]-4-oxo-2- thioxo-1,3-thiazolidin-3-yl}acetic acid |
| 210 | 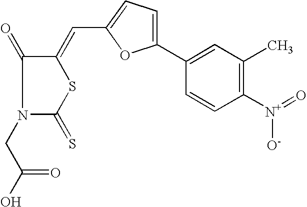 | {(5Z)-5-([5-(3-methyl-4-nitrophenyl)-2- furyl]methylene]-4-oxo-2-thioxo-1,3-thiazolidin-3- yl)acetic acid |
| 211 | 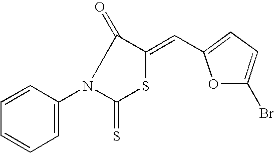 | (5Z)-5-[(5-bromo-2-furyl)methylene]-3-phenyl-2- thioxo-1,3-thiazolidin-4-one |
| 212 | 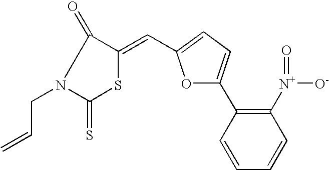 | (5Z)-3-allyl-5-{[5-(2-nitrophenyl)-2- furyl]methylene}-2-thioxo-1,3-thiazolidin-4-one |
| 213 | 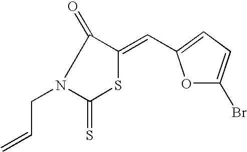 | (5Z)-3-allyl-5-[(5-bromo-2-furyl)methylene]-2- thioxo-1,3-thiazolidin-4-one |
| 214 | 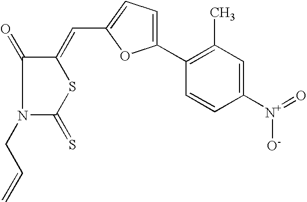 | (5Z)-3-allyl-5-{[5-(2-methyl-4-nitrophenyl)-2- furyl]methylene]-2-thioxo-1,3-thiazolidin-4-one |
| 215 | 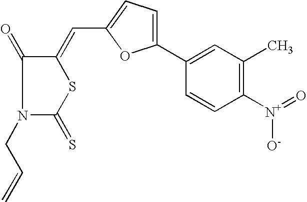 | (5Z)-3-allyl-5-{[5-(3-methyl-4-nitrophenyl)-2- furyl]methylene]-2-thioxo-1,3-thiazolidin-4-one |
| 216 | 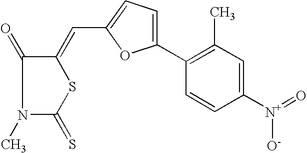 | (5Z)-3-methyl-5-{[5-(2-methyl-4-nitrophenyl)-2- furyl]methylene}-2-thioxo-1,3-thiazolidin-4-one |
| 217 | 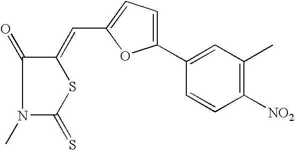 | (5Z)-3-methyl-5-{[5-(3-methyl-4-nitrophenyl)-2- furyl]methylene}-2-thioxo-1,3-thiazolidin-4-one |
| 218 | 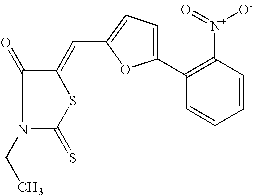 | (5Z)-3-ethyl-5-{[5-(2-nitrophenyl)-2- furyl]methylene}-2-thioxo-1,3-thiazolidin-4-one |
| 219 | 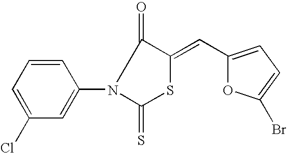 | (5Z)-5-[(5-bromo-2-furyl)methylene]-3-(3- chlorophenyl)-2-thioxo-1,3-thiazolidin-4-one |
| 220 | 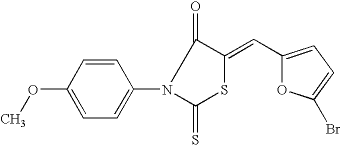 | (5Z)-5-[(5-bromo-2-furyl)methylene]-3-(4- methoxyphenyl)-2-thioxo-1,3-thiazolidin-4-one |
| 221 | 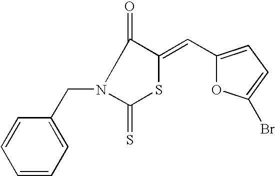 | (5Z)-3-benzyl-5-[(5-iodo-2-furyl)methylene]-2- thioxo-1,3-thiazolidin-4-one |
| 223 | 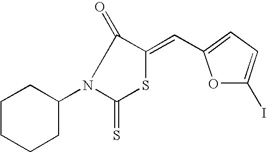 | (5Z)-3-cyclohexyl-5-[(5-iodo-2-furyl)methylene]-2- thioxo-1,3-thiazolidin-4-one |
| 224 | 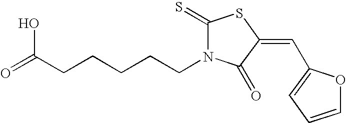 | 6-[(5E)-5-(2-furylmethylene)-4-oxo-2-thioxo-1,3- thiazolidin-3-yl]hexanoic acid |
| 225 | 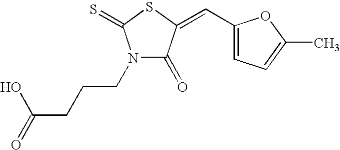 | 4-[(5E)-5-[(5-methyl-2-furyl)methylene]-4-oxo-2- thioxo-1,3-thiazolidin-3-yl}butanoic acid |
| 226 | 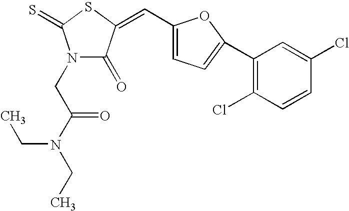 | 2-((5E)-5-{[5-(2,5-dichlorophenyl)-2- furyl]methylene}-4-oxo-2-thioxo-1,3-thiazolidin-3- yl)-N,N-diethylacetamide |
| 227 | 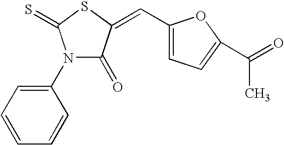 | (5E)-5-[(5-acetyl-2-furyl)methylene]-3-phenyl-2- thioxo-1,3-thiazolidin-4-one |
| 228 | 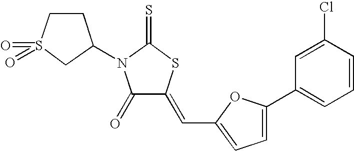 | (5Z)-5-{[5-(3-chlorophenyl)-2-furyl]methylene}-3- (1,1-dioxidotetrahydro-3-thienyl)-2-thioxo-11,3- thiazolidin-4-one |
| 229 | 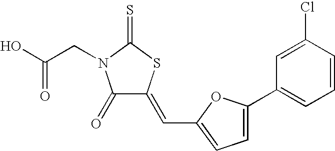 | ((5Z)-5-{[5-(3-chlorophenyl)-2-furyl]methylene}-4- oxo-2-thioxo-1,3-thiazolidin-3-yl)acetic acid |
| 230 | 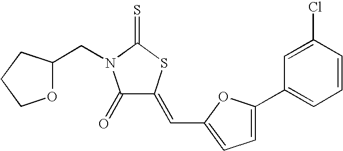 | (5Z)-5-{[5-(3-chlorophenyl)-2-furyl]methylene}-3- (tetrahydrofuran-2-ylmethyl)-2-thioxo-1,3- thiazolidin-4-one |
| 231 | 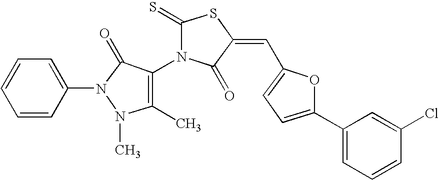 | (5E)-5-{[5-(3-chlorophenyl)-2-furyl]methylene}-3- (1,5-dimethyl-3-oxo-2-phenyl-2,3-dihydro-1H- pyrazol-4-yl)-2-thioxo-1,3-thiazolidin-4-one |
| 232 | 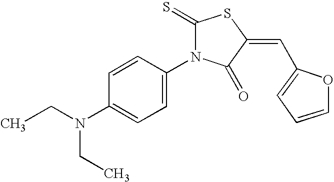 | (5E)-33-[4-(diethylamino)phenyl]-5-(2- furylmethylene)-2-thioxo-1,3-thiazolidin-4-one |
| 233 | 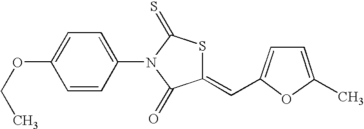 | (5Z)-3-(4-ethoxyphenyl)-5-[(5-methyl-2- furyl)methylene]-2-thioxo-1,3-thiazolidin-4-one |
| 234 | 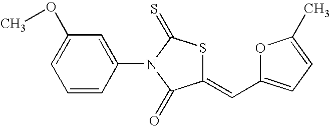 | (5Z)-3-(3-methoxyphenyl)-5-](5-methyl-2- furyl)methylene]-2-thioxo-1,3-thiazolidin-4-one |
| 235 | 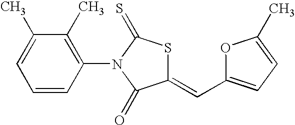 | (5Z)-3-(2,3-dimethylphenyl)-5-[(5-methyl-2- furyl)methylene]-2-thioxo-1,3-thiazolidin-4-one |
| 236 | 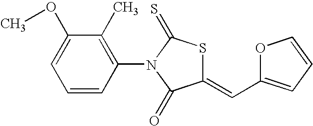 | (5Z)-5-(2-furylmethylene)-3-(methoxyphenyl)-2- thioxo-1,3-thiazolidin-4-one |
| 237 | 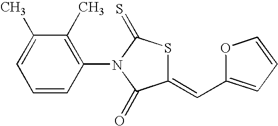 | (5Z)-3-(2,3-dimethylphenyl)-5-(2-furylmethylene)- 2-thioxo-1,3-thiazolidin-4-one |
| 238 | 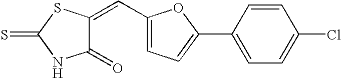 | (5E)-5-{[5-(4-chlorophenyl)-2-furyl]methylene}-2- thioxo-1,3-thiazolidin-4-one |
| 239 | 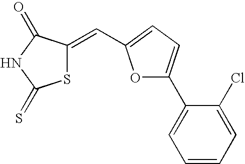 | (5Z)-5-{[5-(2-chlorophenyl)-2-furyl]methylene}-2- thioxo-1,3-thiazolidin-4-one |
| 240 | 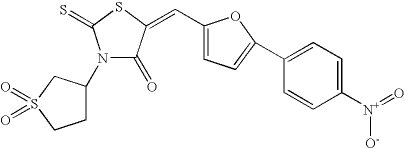 | (5E)-3-(1,1-dioxidotetrahydro-3-thienyl)-5-{[5-(4- nitrophenyl)-2-furyl]methylene}-2-thioxo-1,3- thiazolidin-4-one |
| 241 | 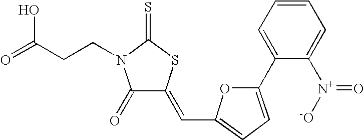 | 3-((5Z)-5-{[5-(2-nitrophenyl)-2-furyl]methylene}-4- oxo-2-thioxo-1,3-thiazolidin-3-yl)propanoic acid |
| 242 | 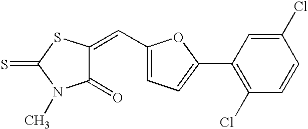 | (5E)-5-{[5-(2,5-dichlorophenyl)-2-furyl]methylene}- 3-methyl-2-thioxo-1,3-thiazolidin-4-one |
| 243 | 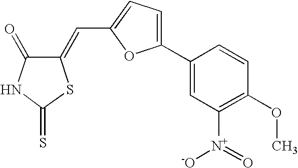 | (5Z)-5-{[5-(4-methoxy-3-nitrophenyl)-2- furyl]methylene}-2-thioxo-1,3-thiazolidin-4-one |
| 244 | 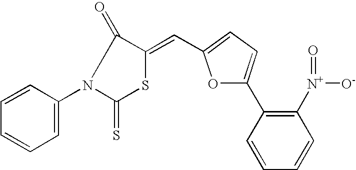 | (5Z)-5-{[5-(2-nitrophenyl)-2-furyl]methylene}-3- phenyl-2-thioxo-1,3-thiazolidin-4-one |
| 245 | 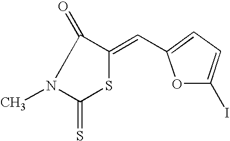 | (5Z)-5-[(5-iodo-2-furyl)methylene]-3-methyl-2- thioxo-1,3-thiazolidin-4-one |
| 246 | 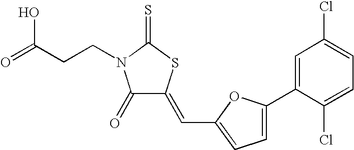 | 3-((5Z)-5-{[5-(2,5-dichlorophenyl)-2- furyl]methylene}-4-oxo-2-thioxo-1,3-thiazolidin-3- yl)propanoic acid |
| 247 | 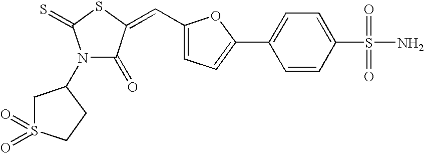 | 4-(5-{(E)-[3-(1,1-dioxidotetrahydro-3-thienyl)-4- oxo-2-thioxo-1,3-thiazolidin-5-ylidene]methyl}-2- furyl)benzenesulfonamide |
| 248 | 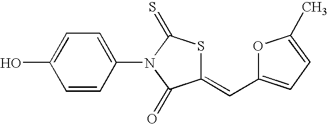 | (5Z)-3-(4-hydroxyphenyl)-5-[(5-methyl-2- furyl)methylene]-2-thioxo-1,3-thiazolidin-4-one |
| 249 | 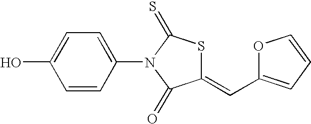 | (5Z)-5-(2-furylmethylene)-3-(4-hydroxyphenyl)-2- thioxo-1,3-thiazolidin-4-one |
| 250 | 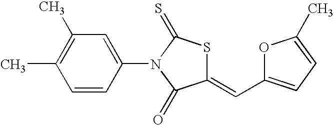 | (5Z)-3-(3,4-dimethylphenyl)-5-[(5-methyl-2- furyl)methylene]-2-thioxo-1,3-thiazolidin-4-one |
| 251 | 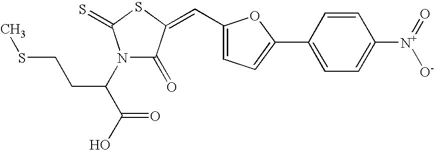 | 4-(methylthio)-2-((5E)-5-{[5-(4-nitrophenyl)-2- furyl]methylene}-4-oxo-2-thioxo-1,3-thiazolidin-3- yl)butanoic acid |
| 252 | 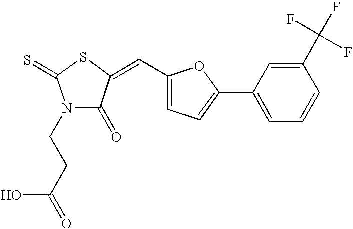 | 3-[(5E)-4-oxo-2-thioxo-5-({5-[3- (trifluoromethyl)phenyl]-2-furyl}methylene}-1,3- thiazolidin-3-yl]propanoic acid |
| 253 | 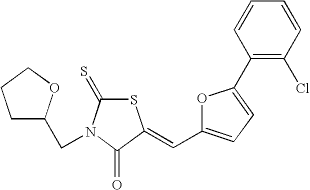 | (5Z)-5-{[5-(2-chlorophenyl)-2-furylmethylene]-3- (tetrahydrofuran-2-ylmethyl)-2-thioxo-1,3- thiazolidin-4-one |
| 254 | 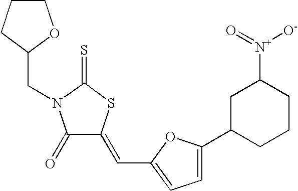 | (5Z)-5-{[5-(3-nitrophenyl)-2-furyl]methylene}-3- (tetrahydrofuran-2-ylmethyl)-2-thioxo-1,3- thiazolidin-4-one |
| 255 | 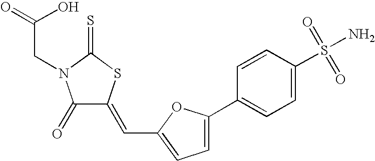 | [(5Z)-5-({5-[4-(aminosulfonyl)phenyl]-2- furyl}methylene)-4-oxo-2-thioxo-1,3-thiazolidin-3- yl]acetic acid |
| 256 | 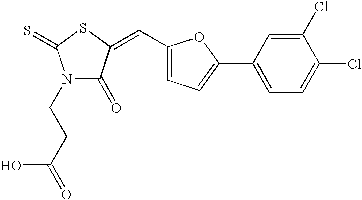 | 3-((5E)-5-{[5-(3,4-dichlorophenyl)-2- furyl]methylene}-4-oxo-2-thioxo-1,3-thiazolidin-3- yl)propanoic acid |
| 257 | 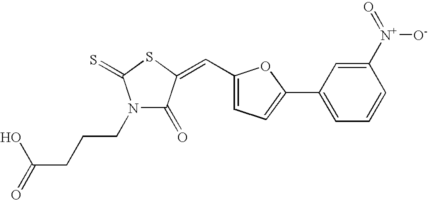 | 4-((5E)-5-{[5-(3-nitrophenyl)-2-furyl]methylene]-4- oxo-2-thioxo-1,3-thiazolidin-3-yl)butanoic acid |
| 258 | 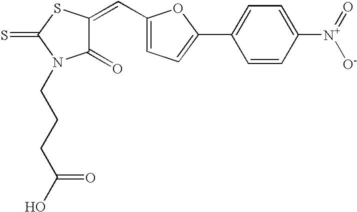 | 4-((5E)-5-{[5-(4-nitrophenyl)-2-furyl]methylene}-4- oxo-2-thioxo-1,3-thiazolidin-3-yl)butanoic acid |
| 259 | 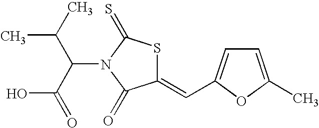 | 3-methyl-2-{(5Z)-5-[(5-methyl-2-furyl)methylene]-4- oxo-2-thioxo-1,3-thiazolidin-3-yl}butanoic acid |
| 260 | 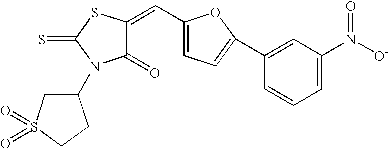 | (5E)-3-(1,1-dioxidotetrahydro-3-thienyl)-5-{[5-(3- nitrophenyl)-2-furyl]methylene}-2-thioxo-1,3- thiazolidin-4-one |
| 261 | 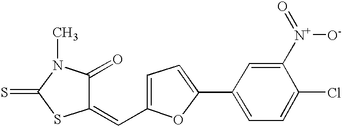 | (5E)-5-{[5-(4-chloro-3-nitrophenyl)-2- furyl]methylene}-3-methyl-2-thioxo-1,3-thiazolidin- 4-one |
| 262 | 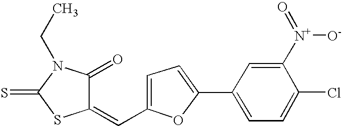 | (5E)-5-{[5-(4-chloro-3-nitrophhenyl)-2- furyl]methylene}-3-ethyl-2-thioxo-1,3-thiazolidin-4- one |
| 263 | 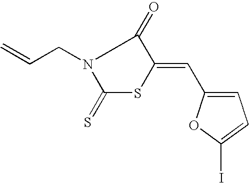 | (5Z)-3-allyl-5-[(5-iodo-2-furyl)methylene]-2-thioxo- 1,3-thiazolidin-4-one |
| 264 | 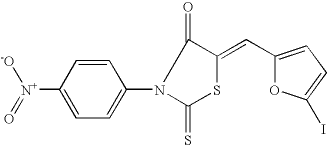 | (5Z)-5-[(5-iodo-2-furyl)methylene]-3-(4- nitrophenyl)-2-thioxo-1,3-thiazolidin-4-one |
| 265 | 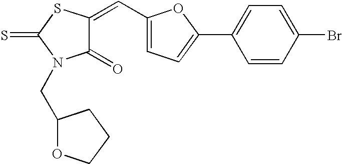 | (5E)-5-{[5-(4-bromophenyl)-2-furyl]methylene}-3- (tetrahydrofuran-2-ylmethyl)-2-thioxo-1,3- thiazolidin-4-one |
| 266 | 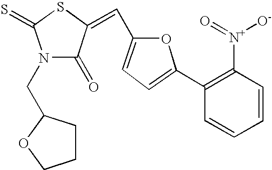 | (5E)-5-{[5-(2-nitrophenyl)-2-furyl]methylene}-3- (tetrahydrofuran-2-ylmethyl)-2-thioxo-1,3- thiazolidin-4-one |
| 267 | 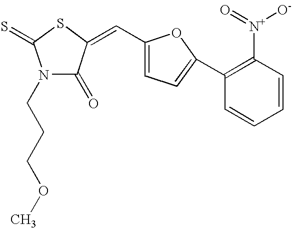 | (5E)-3-(3-methoxypropyl)-5-{[5-(2-nitrophenyl)-2- furyl]methylene}-2-thioxo-1,3-thiazolidin-4-one |
| 268 | 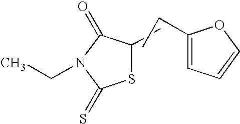 | (5Z)-3-ethyl-5-(2-furylmthylene)-2-thioxo-1,3- thiazolidin-4-one |
| 269 | 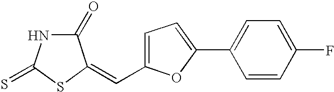 | (5E)-5-{[5-(4-fluorophenyl)-2-furyl]methylene}-2- thioxo-1,3-thiazolidin-4-one |
| 270 | 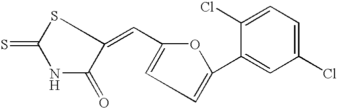 | (5E)-5-{[5-(2,5-dichlorophenyl)-2-furyl]methylene}- 2-thioxo-1,3-thiazolidin-4-one |
| 271 | 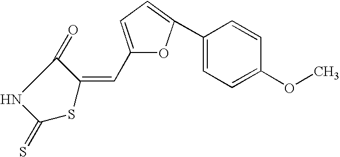 | (5E)-5-{[5-(4-methoxyphenyl)-2-furyl]methylene}-2- thioxo-1,3-thiazolidin-4-one |
| 272 | 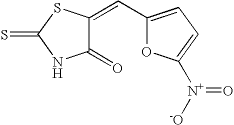 | (5E)-5-[(5-nitro-2-furyl)methylene]-2-thioxo-11,3- thiazolidin-4-one |
| 273 | 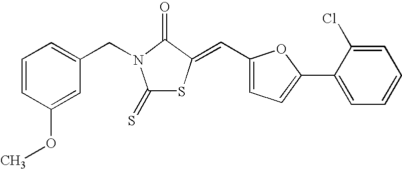 | (5Z)-5-{[5-(2-chlorrophenyl)-2-furyl]methylene}-3- (3-methoxybenzyl)-2-thioxo-1,3-thiazolidin-4-one |
| 274 | 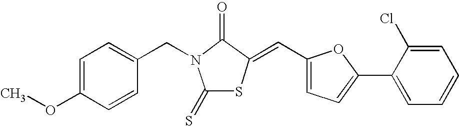 | (5Z)-5-{[5-(2-chlorophenyl)-2-furyl]methylene}-3- (4-methoxybenzyl)-2-thioxo-11,3-thiazolidin-4-one |
| 275 | 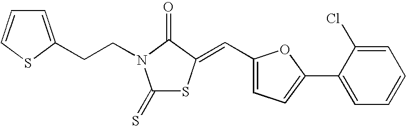 | (5Z)-5-{[5-(2-chlorophenyl)-2-furyl]methylene}-3- [2-(2-thienyl)ethyl]-2-thioxo-1,3-thiazolidin-4-one |
| 276 | 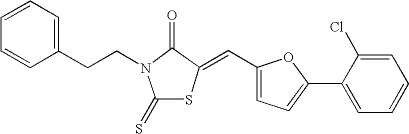 | (5Z)-5-{[5-(2-chlorophenyl)-2-furyl]methylene}-3- (2-phenylethyl)-2-thioxo-1,3-thiazolidin-4-one |
| 277 | 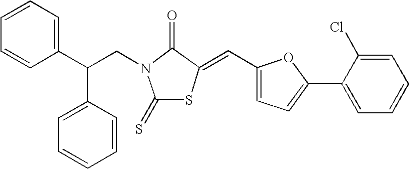 | (5Z)-5-{[5-(2-chlorophenyl)-2-furyl]methylene}-3- (2,2-diphenylethyl)-2-thioxo-1,3-thiazolidin-4-one |
| 278 | 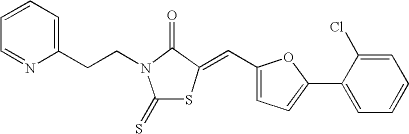 | (5Z)-5-{[5-(2-chlorophenyl)-2-furyl]methylene}-3- (2-pyridin-2-ylethyl)-2-thioxo-1,3-thiazolidin-4-one |
| 279 | 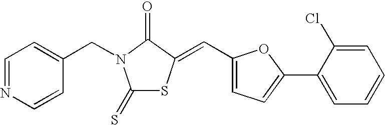 | (5Z)-5-{[5-(2-chlorophenyl)-2-furyl]methylene}-3- (pyridin-4-ylmethyl)-2-thioxo-1,3-thiazolidin-4-one |
| 280 | 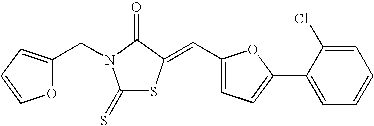 | (5Z)-5-{[5-(2-chlorophenyl)-2-furyl]methylene}-3- (2-furylmethyl)-2-thioxo-1,3-thiazolidin-4-one |
| 281 | 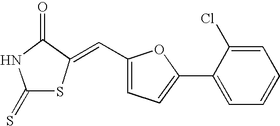 | (5Z)-5-{[5-(2-chlorophenyl)-2-furyl]methylene}- thioxo-1,3-thiazolidin-4-one |
| 282 | 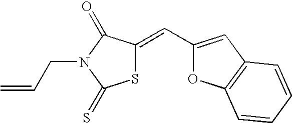 | (5Z)-3-allyl-5-(1-benzofuran-2-ylmethylene)-2- thioxo-1,3-thiazolidin-4-one |
| 283 | 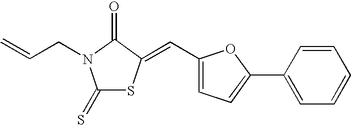 | (5Z)-3-allyl-4-[(5-phenyl-2-furyl)methylene]-2- thioxo-1,3-thiazolidin-4-one |
| 284 | 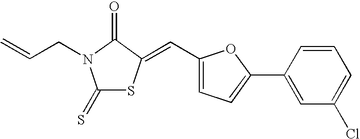 | (5Z)-3-allyl-5-{[5-(3-chlorophenyl)-2- furyl]methylene}-2-thioxo-1,3-thiazolidin-4-one |
| 285 | 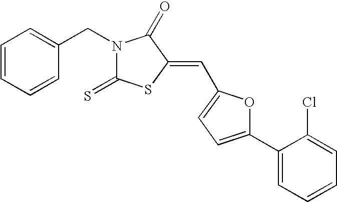 | (5Z)-3-benzyl-5-{[5-(2-chlorophenyl)-2- furyl]methylene}-2-thioxo-1,3-thiazolidin-4-one |
| 286 | 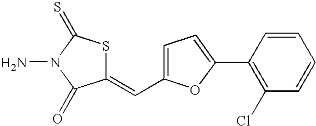 | (5Z)-3-amino-5-{[5-(2-chlorophenyl)-2- furyl]methylene}-2-thioxo-1,3-thiazolidin-4-one |
| 287 | 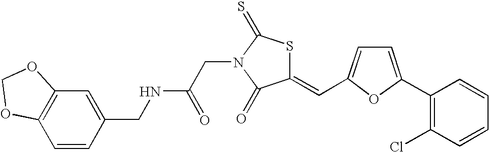 | N-(1,3-benzodioxol-5-ylmethyl)-2-((5Z)-5-{[5-(2- chlorophenyl)-2-furyl]methylene}-4-oxo-2-thioxo- 1,3-thiazolidin-3-yl)acetammide |
| 288 | 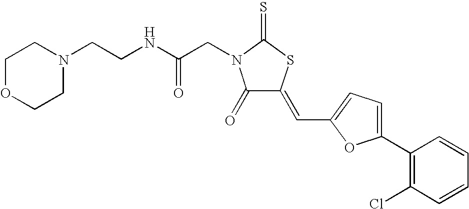 | 2-((5Z)-5-{[5-(2-chlorophenyl)-2-furyl]methylene}- 4-oxo-2-thioxo-1,3-thiazolidin-3-yl)-N-(2-morpholin- 4-ylethyl)acetamide |
| 289 | 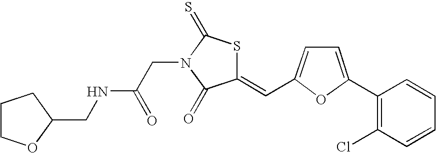 | 2-((5Z)-5-{[5-(2-chlorophenyl)-2-furyl]methylene}- 4-oxo-2-thioxo-1,3-thiazolidin-3-yl)-N- (tetrahydrofuran-2-ylmethyl)acetamide |
| 290 | 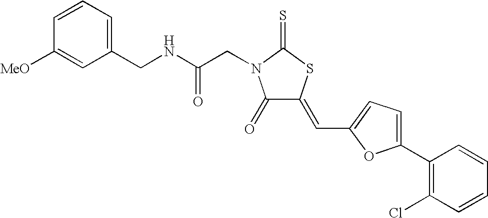 | 2-((5Z)-5-{[5-(2-chlorophenyl)-2-furyl]methylene}- 4-oxo-2-thioxo-1,3-thiazolidin-3-yl)-N-(3- methoxybenzyl)acetamide |
| 291 | 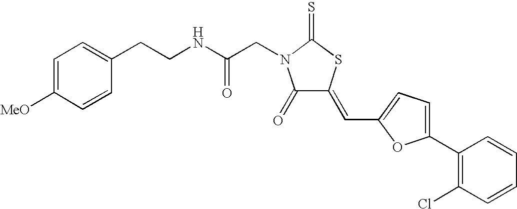 | 2-((5Z)-5-{[5-(2-chlorophenyl)-2-furyl]methylene}- 4-oxo-2-thioxo-1,3-thiazolidin-3-yl)-N-[2-(4- methoxyphenyl)ethyl]acetamide |
| 292 | 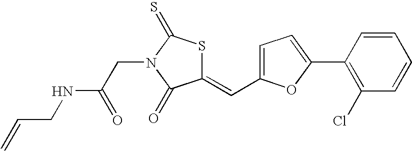 | N-allyl-2-((5Z)-5-{[5-(2-chlorophenyl)-2- furyl]methylene}-4-oxo-2-thioxo-1,3-thiazolidin-3- yol)acetamide |
| 293 | 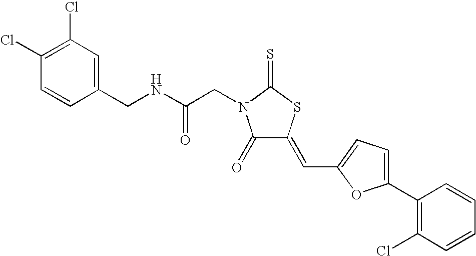 | 2-((5Z)-5-{[5-(2-chlorophenyl)-2-furyl]methylene}- 4-oxo-2-thioxo-1,3-thiazolidin-3-yl)-N-(3,4- dichlorobenzyl)acetamide |
| 294 | 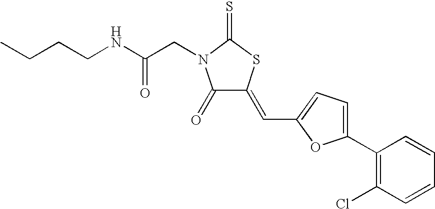 | N-butyl-2-((5Z)-5-{[5-(2-chlorophenyl)-2- furyl]methylene}-4-oxo-2-thioxo-1,3-thiazolidin-3- yl)acetamide |
| 295 | 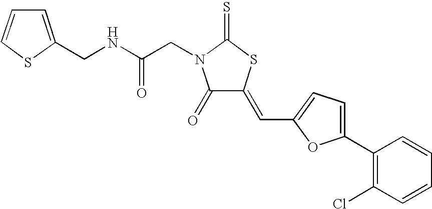 | 2-((5Z)-5-{[5-(2-chlorophenyl)-2-furyl]methylene}- 4-oxo-2-thioxo-1,3-thiazolidin-3-yl)-N-(2- thienylmethyl)acetamide |
| 296 | 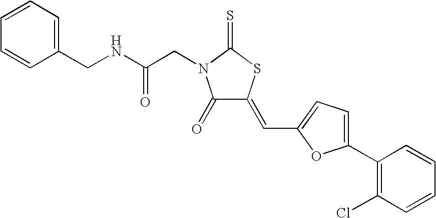 | N-benzyl-2-((5Z)-5-{[5-(2-chlorophenyl)-2- furyl]methylene}-4-oxo-2-thioxo-1,3-thiazolidin-3- yl)acetamide |
| 297 | 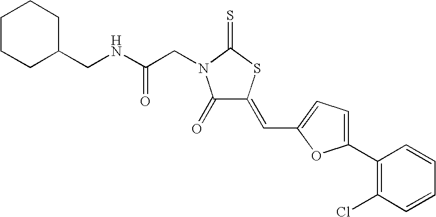 | 2-((5Z)-5-{[5-(2-chlorophenyl)-2-furyl]methylene}- 4-oxo-2-thioxo-1,3-thiazolidin-3-yl)-N- (cyclohexylmethyl)acetamide |
| 298 | 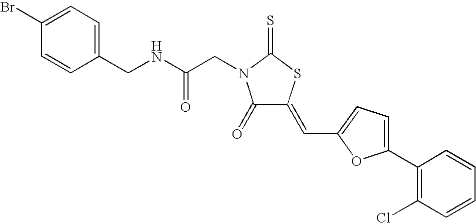 | N-(4-bromobenzyl)-2-((5Z)-5-{[5-(2-chlorophenyl)- 2-furyl]methylene}-4-oxo-2-thioxo-1,3-thiazolidin- 3-yl)acetamide |
| 299 | 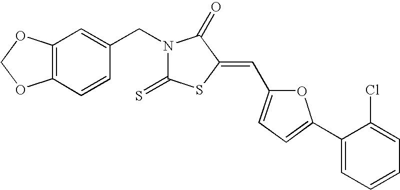 | (5Z)-3-(1,3-benzodioxol-5-ylmethyl)-5-{[5-(2- chlorophenyl)-2-furyl]methylene}-2-thioxo-1,3- thiazolidin-4-one |
| 300 | 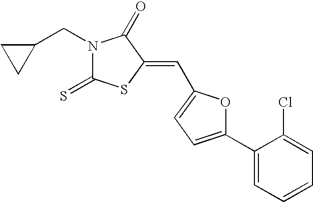 | (5Z)-5-{[5-(2-chlorophenyl)-2-furyl]methylene}-3- (cyclopropylmethyl)-2-thioxo-1,3-thiazolidin-4-one |
| 301 | 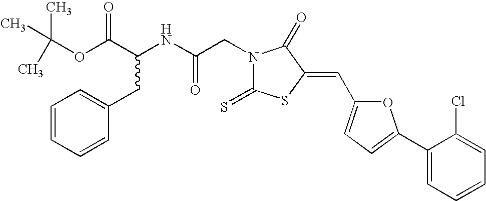 | tert-butyl N-[((5Z)-5-{[5-(2-chlorophenyl)-2- furyl]methylene}-4-oxo-2-thioxo-1,3-thiazolidin-3- yl)acetyl]phenylalaninate |
| 302 | 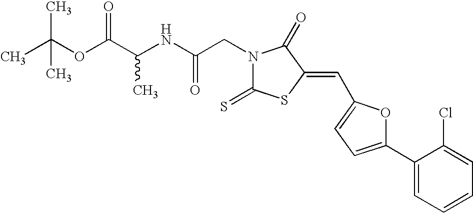 | tert-butyl N-[((5Z)-5-{[5-(2-chlorophenyl)-2- furyl]methylene}-4-oxo-2-thioxo-1,3-thiazolidin-3- yl)acetyl]alaninate |
| 303 | 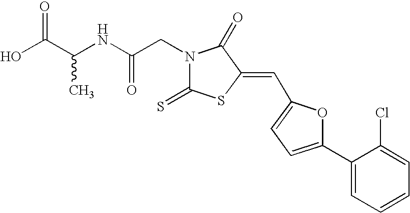 | N-[((5Z)-5-{[5-(2-chlorophenyl)-2-furyl]methylene}- 4-oxo-2-thioxo-1,3-thiazolidin-3-yl}acetyl]alanine |
| 304 | 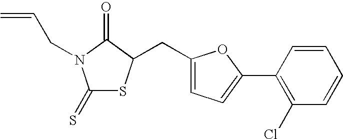 | 3-allyl-5-{[5-(2-chlorophenyl)-2-furyl]methyl}-2- thioxo-1,3-thiazolidin-4-one |
| 305 | 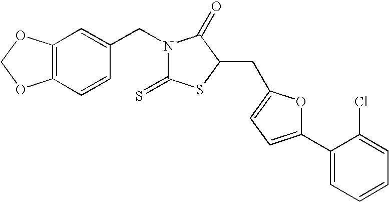 | 3-(1,3-benzodioxol-5-ylmethyl)-5-{[5-(2- chlorophenyl)-2-furyl]methyl}-2-thioxo-1,3- thiazolidin-4-one |
| 306 | 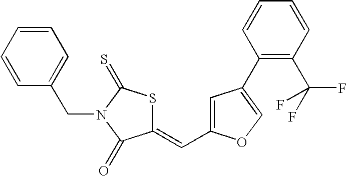 | (5Z)-3-benzyl-5-{[5-(2-trifluoromethylphenyl)-2- furyl]methylene}-2-thioxo-1,3-thiazolidin-4-one |
| 307 | 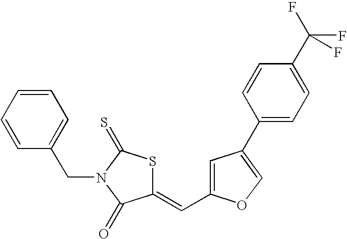 | (5Z)-3-benzyl-5-{[5-(4-trifluoromethylphenyl)-2- furyl]methylene}-2-thioxo-1,3-thiazolidin-4-one |

While particular geometric isomers (i.e., E or Z) are displayed throughout this specification, the invention also comprises the E or Z geometric isomers and mixtures thereof of all of the compounds of paragraphs [0016]-[0020], as well as the compounds disclosed in the table in paragraph [0026]. The E and Z geometric isomers can be interconverted by photolysis, photo irradiation or exposure to free radicals. See, e.g., Ishida et al., Tetrahedron Lett. 30, 959 (1989). Exposure to certain solvents, e.g., DMSO, will facilitate conversion of an E isomer to the Z form.

The compounds in the table above can be prepared using art recognized methods. All of the compounds in this application were named using ChemDraw Ultra version 6.0.2, which is available through Cambridgesoft.com, 100 Cambridge Park Drive, Cambridge, Mass. 02140, Namepro version 5.09, which is available from ACD labs, 90 Adelaide Street West, Toronto, Ontario, M5H, 3V9, Canada, or were derived therefrom.

For simplicity, chemical moieties are defined and referred to throughout primarily as univalent chemical moieties (e.g., alkyl, aryl, etc.). Nevertheless, such terms are also used to convey corresponding multivalent moieties under the appropriate structural circumstances clear to those skilled in the art. For example, while an “alkyl” moiety generally refers to a monovalent radical (e.g. CH₃CH₂—), in certain circumstances a bivalent linking moiety can be “alkyl,” in which case those skilled in the art will understand the alkyl to be a divalent radical (e.g., —CH₂CH₂—), which is equivalent to the term “alkylene.” (Similarly, in circumstances in which a divalent moiety is required and is stated as being “aryl,” those skilled in the art will understand that the term “aryl” refers to the corresponding divalent moiety, arylene.) All atoms are understood to have their normal number of valences for bond formation (i.e., 4 for carbon, 3 for N, 2 for O, and 2, 4, or 6 for S, depending on the oxidation state of the S). On occasion a moiety may be defined, for example, as (A)a B, wherein a is 0 or 1. In such instances, when a is 0 the moiety is B and when a is 1 the moiety is A B. Also, a number of moieties disclosed herein exist in multiple tautomeric forms, all of which are intended to be encompassed by any given tautomeric structure. Other stereochemical forms of the compounds of the invention are also encompassed including but not limited to enantiomers, diastereomers, and other isomers such as rotamers.

For simplicity, when a substituent can be of a particular chemical class differing by the number of atoms or groups of the same kind in the moiety (e.g., alky, which can be C₁, C₂, C₃, etc.), the number of repeated atoms or groups is represented by a range (e.g., C₁-C₆ alkyl). In such instances each and every number in that range and all sub ranges are specifically contemplated. Thus, C₁-C₃ alkyl means C₁, C₂, C₃, C₁₋₂, C₁₋₃, and C₂₋₃ alkyl.

In addition to individual preferred embodiments of each substituent defined herein, the invention also comprises all combinations of preferred substituents.

The term “alkyl” as employed herein refers to straight and branched chain aliphatic groups having from 1 to 30 carbon atoms, preferably 1 to 15 carbon atoms, more preferably 1 to 6 carbon atoms, which is optionally substituted with one, two or three substituents. Unless otherwise specified, the alkyl group may be saturated, unsaturated, or partially unsaturated. As used herein, therefore, the term “alkyl” is specifically intended to include alkenyl and alkynyl groups, as well as saturated alkyl groups, unless expressly stated otherwise. Preferred alkyl groups include, without limitation, methyl, ethyl, propyl, isopropyl, butyl, tert butyl, isobutyl, pentyl, hexyl, vinyl, allyl, isobutenyl, ethynyl, and propynyl.

As employed herein, a “substituted” alkyl, cycloalkyl, aryl, or heterocyclic group is one having between one and about four, preferably between one and about three, more preferably one or two, non hydrogen substituents. Suitable substituents include, without limitation, halo, hydroxy, nitro, haloalkyl, alkyl, alkaryl, aryl, aralkyl, alkoxy, aryloxy, amino, acylamino, alkylcarbamoyl, arylcarbamoyl, aminoalkyl, alkoxycarbonyl, carboxy, hydroxyalkyl, alkanesulfonyl, arenesulfonyl, alkanesulfonamido, arenesulfonamido, aralkylsulfonamido, alkylcarbonyl, acyloxy, cyano, and ureido groups.

The term “cycloalkyl” as employed herein includes saturated and partially unsaturated cyclic hydrocarbon groups having 3 to 12, preferably 3 to 8 carbons, wherein the cycloalkyl group additionally is optionally substituted. Preferred cycloalkyl groups include, without limitation, cyclopropyl, cyclobutyl, cyclopentyl, cyclopentenyl, cyclohexyl, cyclohexenyl, cycloheptyl, and cyclooctyl.

The term “hydrocarbyl” as employed herein includes all alkyl moieties and all cycloalkyl moieties (both as defined above), each alone or in combination. Thus, for example, hydrocarbyl includes methyl, ethyl, propyl, n-butyl, isobutyl, cyclopropyl, cyclohexyl, cyclopropyl—CH₂, cyclohexyl-(CH₂)₃, etc.

An “aryl” group is a C₆-C₁₄ aromatic moiety comprising one to three aromatic rings, which is optionally substituted. Preferably, the aryl group is a C₆-C₁₀ aryl group. Preferred aryl groups include, without limitation, phenyl, naphthyl, anthracenyl, and fluorenyl. An “aralkyl” or “arylalkyl” group comprises an aryl group covalently linked to an alkyl group, either of which may independently be optionally substituted or unsubstituted. Preferably, the aralkyl group is C₁-C₆ alkyl (C₆-C₁₀)aryl, including, without limitation, benzyl, phenethyl, and naphthylmethyl. An “alkaryl” or “alkylaryl” group is an aryl group having one or more alkyl substituents. Examples of alkaryl groups include, without limitation, tolyl, xylyl, mesityl, ethylphenyl, tert butylphenyl, and methylnaphthyl.

A “heterocyclic” group (or “heterocyclyl) is a non-aromatic mono-, bi-, or tricyclic structure having from about 3 to about 14 atoms, wherein one or more atoms are selected from the group consisting of N, O, and S. One ring of a bicyclic heterocycle or two rings of a tricyclic heterocycle may be aromatic, as in indan and 9,10-dihydro anthracene. The heterocyclic group is optionally substituted on carbon with oxo or with one of the substituents listed above. The heterocyclic group may also independently be substituted on nitrogen with alkyl, aryl, aralkyl, alkylcarbonyl, alkylsulfonyl, arylcarbonyl, arylsulfonyl, alkoxycarbonyl, aralkoxycarbonyl, or on sulfur with oxo or lower alkyl. Preferred heterocyclic groups include, without limitation, epoxy, aziridinyl, tetrahydrofuranyl, pyrrolidinyl, piperidinyl, piperazinyl, thiazolidinyl, oxazolidinyl, oxazolidinonyl, and morpholino.

In certain preferred embodiments, the heterocyclic group is a heteroaryl group. As used herein, the term “heteroaryl” refers to groups having 5 to 14 ring atoms, preferably 5, 6, 9, or 10 ring atoms; having 6, 10, or 14 pi electrons shared in a cyclic array; and having, in addition to carbon atoms, between one and about three heteroatoms selected from the group consisting of N, O, and S. Preferred heteroaryl groups include, without limitation, thienyl, benzothienyl, furyl, benzofuryl, dibenzofuryl, pyrrolyl, imidazolyl, pyrazolyl, pyridyl, pyrazinyl, pyrimidinyl, indolyl, quinolyl, isoquinolyl, quinoxalinyl, tetrazolyl, oxazolyl, thiazolyl, and isoxazolyl.

In certain other preferred embodiments, the heterocyclic group is fused to an aryl or heteroaryl group. Examples of such fused heterocycles include, without limitation, tetrahydroquinolinyl and dihydrobenzofuranyl. Additional preferred heterocyclyls and heteroaryls include, but are not limited to, acridinyl, azocinyl, benzimidazolyl, benzofuranyl, benzothiofuranyl, benzothiophenyl, benzoxazolyl, benzodioxolyl, benzthiazolyl, benztriazolyl, benztetrazolyl, benzisoxazolyl, benzisothiazolyl, benzimidazolinyl, carbazolyl, 4aH-carbazolyl, carbolinyl, chromanyl, chromenyl, cinnolinyl, decahydroquinolinyl, 2H,6H-1,5,2-dithiazinyl, dihydrofuro[2,3 b]tetrahydrofuran, furanyl, furazanyl, imidazolidinyl, imidazolinyl, 1H-indazolyl, indolenyl, indolinyl, indolizinyl, 3H-indolyl, isobenzofuranyl, isochromanyl, isoindazolyl, isoindolinyl, isoindolyl, isothiazolyl, methylenedioxyphenyl, morpholinyl, naphthyridinyl, octahydroisoquinolinyl, oxadiazolyl, 1,2,3-oxadiazolyl, 1,2,4-oxadiazolyl, 1,2,5-oxadiazolyl, 1,3,4-oxadiazolyl, oxazolidinyl, oxazolidinyl, pyrimidinyl, phenanthridinyl, phenanthrolinyl, phenazinyl, phenothiazinyl, phenoxathiinyl, phenoxazinyl, phthalazinyl, piperazinyl, piperidinyl, piperidonyl, 4-piperidonyl, piperonyl, pteridinyl, purinyl, pyranyl, pyrazolidinyl, pyrazolinyl, pyridazinyl, pyridooxazole, pyridoimidazole, pyridothiazole, pyridinyl, pyrrolidinyl, pyrrolinyl, 2H-pyrrolyl, quinazolinyl, 4H-quinolizinyl, quinuclidinyl, tetrahydroisoquinolinyl, 6H-1,2,5-thiadiazinyl, 1,2,3-thiadiazolyl, 1,2,4-thiadiazolyl, 1,2,5-thiadiazolyl, 1,3,4-thiadiazolyl, thianthrenyl, thienyl, thienothiazolyl, thienooxazolyl, thienoimidazolyl, thiophenyl, triazinyl, 1,2,3-triazolyl, 1,2,4-triazolyl, 1,2,5-triazolyl, 1,3,4-triazolyl, and xanthenyl.

A moiety that is substituted is one in which one or more hydrogens have been independently replaced with another chemical substituent. As a non limiting example, substituted phenyls include 2-fluorophenyl, 3,4-dichlorophenyl, 3-chloro-4-fluorophenyl, 2-fluoro-3-propylphenyl. As another non limiting example, substituted n octyls include 2,4-dimethyl-5-ethyloctyl and 3-cyclopentyloctyl. Included within this definition are methylenes (—CH₂—) substituted with oxygen to form carbonyl (—CO).

Unless otherwise stated, as employed herein, when a moiety (e.g., cycloalkyl, hydrocarbyl, aryl, heteroaryl, heterocyclic, urea, etc.) is described as “optionally substituted” it is meant that the group optionally has from one to four, preferably from one to three, more preferably one or two, non hydrogen substituents. Suitable substituents include, without limitation, halo, hydroxy, oxo (e.g., an annular —CH— substituted with oxo is —C(O)—) nitro, halohydrocarbyl, hydrocarbyl, aryl, aralkyl, alkoxy, aryloxy, amino, acylamino, alkylcarbamoyl, arylcarbamoyl, aminoalkyl, acyl, carboxy, hydroxyalkyl, alkanesulfonyl, arenesulfonyl, alkanesulfonamido, arenesulfonamido, aralkylsulfonamido, alkylcarbonyl, acyloxy, cyano, and ureido groups. Preferred substituents, which are themselves not further substituted (unless expressly stated otherwise) are:
(a) halo, cyano, oxo, carboxy, formyl, nitro, amino, amidino, guanidino,
(b) C₁-C₅ alkyl or alkenyl or arylalkyl imino, carbamoyl, azido, carboxamido, mercapto, hydroxy, hydroxyalkyl, alkylaryl, arylalkyl, C₁-C₈ alkyl, C₁-C₈ alkenyl, C₁-C₈ alkoxy, C₁-C₈ alkoxycarbonyl, aryloxycarbonyl, C₂-C₈ acyl, C₂-C₈ acylamino, C₁-C₈ alkylthio, arylalkylthio, arylthio, C₁-C₈ alkylsulfinyl, arylalkylsulfinyl, arylsulfinyl, C₁-C₈ alkylsulfonyl, arylalkylsulfonyl, arylsulfonyl, C₀-C₆ N-alkyl carbamoyl, C₂-C₁₅ N,N-dialkylcarbamoyl, C₃-C₇ cycloalkyl, aroyl, aryloxy, arylalkyl ether, aryl, aryl fused to a cycloalkyl or heterocycle or another aryl ring, C₃-C₇ heterocycle, or any of these rings fused or spiro fused to a cycloalkyl, heterocyclyl, or aryl, wherein each of the foregoing is further optionally substituted with one more moieties listed in (a), above; and
(c) —(CH₂)ₛ NR₃₀R₃₁, wherein s is from 0 (in which case the nitrogen is directly bonded to the moiety that is substituted) to 6, and R₃₀ and R₃₁ are each independently hydrogen, cyano, oxo, carboxamido, amidino, C₁-C₈ hydroxyalkyl, C₁-C₃ alkylaryl, aryl C₁-C₃ alkyl, C₁-C₈ alkyl, C₁-C₈ alkenyl, C₁-C₈ alkoxy, C₁-C₈ alkoxycarbonyl, aryloxycarbonyl, aryl C₁-C₃ alkoxycarbonyl, C₂-C₈ acyl, C₁-C₈ alkylsulfonyl, arylalkylsulfonyl, arylsulfonyl, aroyl, aryl, cycloalkyl, heterocyclyl, or heteroaryl, wherein each of the foregoing is further optionally substituted with one more moieties listed in (a), above; or
   R₃₀ and R₃₁ taken together with the N to which they are attached form a heterocyclyl or heteroaryl, each of which is optionally substituted with from 1 to 3 substituents from (a), above.

The term “haloaen” or “halo” as employed herein refers to chlorine, bromine, fluorine, and iodine.

As herein employed, the term “acyl” refers to an alkylcarbonyl or arylcarbonyl substituent.

The term “acylamino” refers to an amide group attached at the nitrogen atom. The term “carbamoyl” refers to an amide group attached at the carbonyl carbon atom. The nitrogen atom of an acylamino or carbamoyl substituent may be additionally substituted. The term “sulfonamido” refers to a sulfonamide substituent attached by either the sulfur or the nitrogen atom. The term “amino” is meant to include NH₂, alkylamino, arylamino, and cyclic amino groups.

### General Synthetic Procedure

The compounds of the invention can be prepared using general synthetic procedures. The starting components are readily prepared from carboxylic acids, aldehydes, alkyls, benzene and phenol to a variety of substitutions can be made according to procedures well known to those skilled in the art and commercially available.

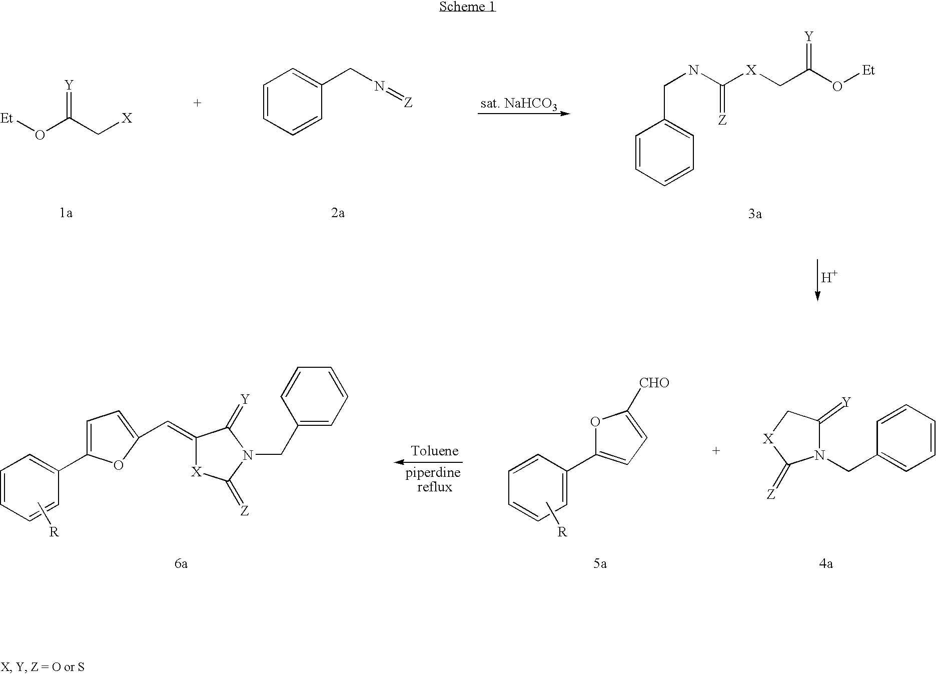

The compounds of the invention can be prepared according to Scheme 1. Scheme 1 illustrates only one way to prepare the compounds of the invention and is not meant to be limiting in any way. One skilled in the art would recognize that to obtain the compounds of the invention, reactant compounds 2a and 5a can be replaced with suitable compounds that have a variety of substituents in the phenyl and furanyl portions. The example below serves to illustrate this point.

### EXAMPLE 1

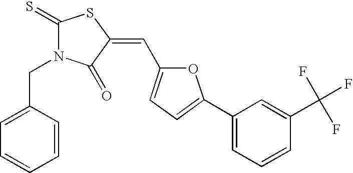

### (5E)-3-benzyl-2-thioxo-5-({5-[3-(trifluoromethyl)phenyl]-2-furyl}methylene)-1,3-thiazolidin-4-one

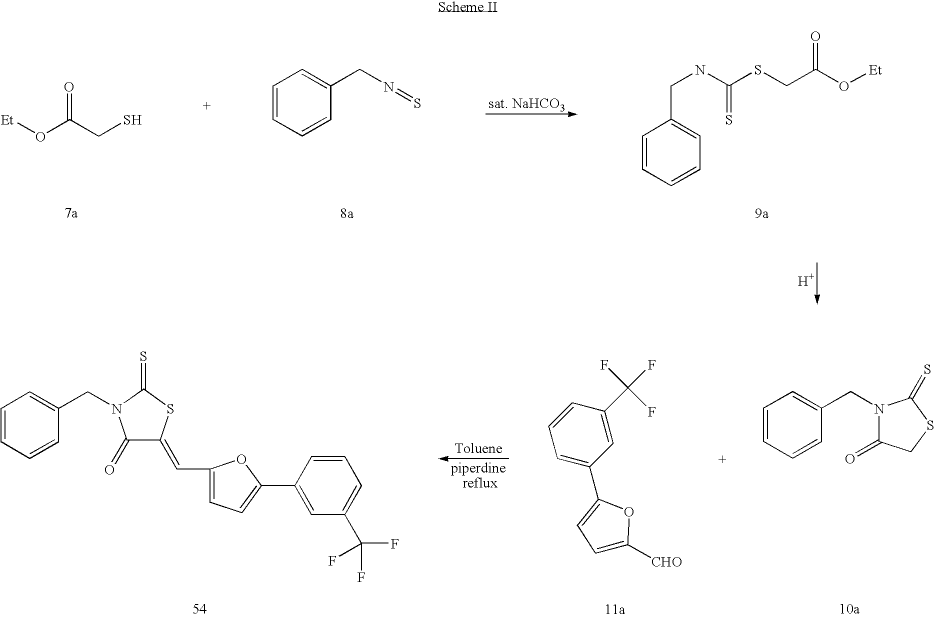 Step 1. Synthesis of Benzyl Rhodanine (10a)

To a mixture of 10 mmol (1.1 mL; 1.2 g) of ethylthio glycolate (7a) and 11 mmole (1.64 g) of benzyl isothiocyanate (8a) was added 26 mL of saturated aqueous sodium bicarbonate. The reaction mixture was stirred at 40° C. for 3 hrs. About 5 mL of methanol was added to enhance solubility. The LC/MS analysis indicated two peaks: the major (85%) corresponded to the desired rhodanine (10a) and the minor peak was that of the uncyclized adduct (9a). The reaction mixture was treated with water and neutralized by addition of acetic acid. The aqueous mixture was extracted with ethyl acetate. The combined organic layers were concentrated to a volume of 10 mL, and 2 mL of acetic acid was added to this. The resulting mixture was heated at 50° C. overnight. Analysis by TLC showed one spot. The product was further purified by column chromatography using silica-gel and 35% ethyl acetate:hexane mixture as the mobile phase. The fractions corresponding to compound 10a were combined to give 2.18 g of pale reddish-yellow needles (yield=98%). ¹H NMR (CDCl₃) □ 3.972 (s, 2H); 5.180 (s, 2H); 7.28 (m, 3H); 7.405 (m, 2H). MS (ES⁻); 222.03 (M−1).

Step 2. Synthesis of Title Compound

To 1.52 g (0.65 mmole) of benzyl rhodanine (10a) was added 30 mL of toluene, 1.56 g (0.65 mmole) of 5-(3-trifluoromethylphenyl)furan-2-carboxaldehyde (11a), and 0.8 mL of piperidine. The mixture was heated under reflux for 4 hours, and the reaction was monitored by TLC. At the end of the 4 hours, the TLC analysis showed no trace of the starting materials. The reaction mixture was allowed to cool and a bright yellow solid formed which was filtered and washed with hexane. The product was further purified by column chromatography using silica-gel and 40% ethyl acetate:hexane mixture as the mobile phase. Yield was 2.6 g (86%). ¹H NMR: (CDCl₃) □ 5.335 (s, 2H); 6.928-6.961 (dd, 2H, J=3.4 Hz); 7.265-7.35 (m, 3H); 7.449-7.488 (m, 3H); 7.613-7.633 (m, 2H); 7.934 (br.s, 1H); 9.945-8.15 (m, 1H). MS; ES+446.21 (M+1).

### EXAMPLE 2

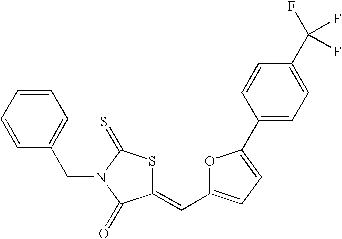

### (5Z)-3-benzyl-5-{[5-(4-trifluoromethylphenyl)-2-furyl]methylene}-2-thioxo-1,3-thiazolidin-4-one

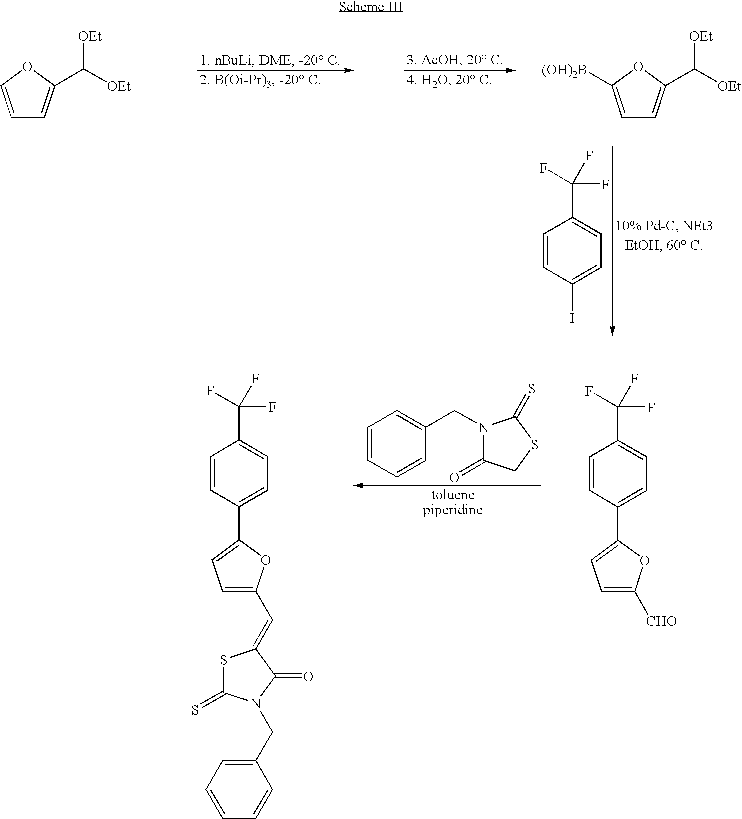

### 1. 5-(Diethoxymethyl)-2-furylboronic acid

To a solution of 2-(diethoxymethyl) furan 16.9 ml 100 mmol) in 150 ml of DME at −20° C., was added 120 mmol of n-BuLi in hexanes dropwise so that the temperature remains below −15° C. The reaction is stirred for a further two hours at −20° C. Triisopropylborate (22.7 ml, 120 m.mol) was then added. The reaction mixture was then allowed to warm up to room temperature. 7.5 mL of acetic acid was then added to the reaction mixture followed by addition of 10 ml of water. The solution was used directly in the next step.

### 2. 5-[4-(Trifluromethyl)phenyl]-2-furaldehyde

To (20 ml, 5 mmol) of the crude boronic acid solution was added (544 mg, 2 mmol) of 4-iodo benzotrifluoride followed by addition of 7 ml of ethanol, 0.6 ml of triethylamine and 54 mg of 10% Pd/C. The reaction mixture was stirred at 60° C. until it was complete by HPLC. The reaction mixture was cooled and filteredand washed with DME till filtrate was colorless. The filtrate was treated with 10 ml of water and 0.8 ml of trifluoroacetic acid and stirred to remove the acetal group. The resulting solution was washed with brine and saturated sodium bicarbonate solutions. The organic layer was dried and solvent evaporated to yield the crude product which was purified by column chromatography using ethyl acetate:hexane 1:4 mixture. The appropriate fractions were combined and evaporated to yield 378 mg of the product 78% yield.

### 3. (5Z)-3-benzyl-5-{[5-(4-trifluoromethylphenyl)-2-furyl]methylene}-2-thioxo-1,3-thiazolidin-4-one

To 44.6 mg of benzylrhodanine was added 48 mg of 5-[4-Trifluromethyl)phenyl]-2-furaldehyde and 10 ml of toluene and 0.1 ml of piperidine. The mixture was refluxed for four hours when an examination of TLC indicated that starting material had been consumed. The reaction mixture was cooled, the solid formed was filtered and washed several times with hexane and dried to yield 82 mg 91% of pure product.

### EXAMPLE 3

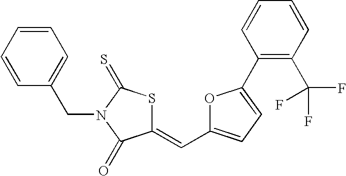

### (5Z)-3-benzyl-5-{[5(2-trifluoromethylphenyl)-2-furyl]methylene}-2-thioxo-1,3-thiazolidin-4-one

To 44.6 mg (0.2 mmol) of benzyl rhodanine was added 48 mg (0.2 mmol) of 5-[2-(trifluoromethyl)phenyl]-2-furaldehyde and 10 mL of toluene. 0.1 ml of piperidine was added to this mixture and the reaction mixture refluxed for four hours. Examination of TLC at this time showed that the reaction was complete. The reaction mixture was cooled. The solid formed was filtered off and the washed several times with hexane. The reaction yielded 80.1 mg (90%) yield of R911572. ¹H NMR: (CDCl₃) δ 5.324 (s, 2H); 6.945 (br.s, 2H); 7.345-7.268 (m, 3H); 7.547-7.438 (m, 4H); 7.675-7.726 (t, 1H, J=7.5 Hz); 7.781-7.7807 (d, 1H, J=7.8 Hz); 7.9267.900 (d, 1H, J=7.8 Hz). MS; Ek⁺ 445.95 (M+1)

### Pharmaceutical Compositions

In a second aspect, the invention provides pharmaceutical compositions comprising an inhibitor of ubiquitination according to the invention and a pharmaceutically acceptable carrier, excipient, or diluent. Suitable excipients are described in “Handbook of Pharmaceutical Excipients,” 4^{th} Edition, Rowe, R. C., Sheskey, P. J., and Weller, P. J., editors, American Pharmaceutical Association, Chicago, Ill. (2003), which is incorporated by reference in its entirety. Compounds of the invention may be formulated by any method well known in the art and may be prepared for administration to the patient by any route, including, without limitation, parenteral, oral, sublingual, subcutaneous, intravenous, intraperitoneal, intramuscular, intrapulmonary, vaginal, rectal, intraocular, transdermal, topical, intranasal, intratracheal, or intrarectal. In some instances, the compounds of the invention are administered directly as a solution or spray. In certain preferred embodiments, compounds of the invention are administered intravenously in a hospital setting. In certain other preferred embodiments, administration may preferably be by the oral route.

The characteristics of the carrier will depend on the route of administration. As used herein, the term “pharmaceutically acceptable” means a non-toxic material that is compatible with a biological system such as a cell, cell culture, tissue, or organism, and that does not interfere with the effectiveness of the biological activity of the active ingredient(s). Thus, pharmaceutical compositions according to the invention may contain, in addition to the inhibitor, carrier proteins (for example, such as serum albumin), diluents, fillers (for example microcrystalline cellulose, lactose, corn and other starches), binding agents, sweeteners and flavoring agents, coloring agents, polyethylene glycol, salts, buffers, stabilizers, solubilizers, flavors, dyes and other materials well known in the art. The preparation of pharmaceutically acceptable formulations is described in many well known references to one skilled in the art, for example, Remington's Pharmaceutical Sciences, 18th Edition, ed. A. Gennaro, Mack Publishing Co., Easton, Pa., 1990.

As used herein, the term pharmaceutically acceptable salts refers to salts and complexes that retain the desired biological activity of the compounds of the invention and exhibit minimal or no undesired toxicological effects. Pharmaceutically acceptable salts include both the acid and base addition salts. Examples of acid salts include, but are not limited to acid addition salts formed with inorganic acids (for example, hydrochloric acid, hydrobromic acid, sulfuric acid, phosphoric acid, nitric acid, and the like), and salts formed with organic acids such as acetic acid, propionic acid, glycolic acid, pyruvic acid, oxalic acid, maleic acid, malonic acid, fumaric acid, tartaric acid, citric acid, cinnamic acid, mandelic acid, methanesulfonic acid, ethanesulfonic acid, p-toluenesulfonic acid, salicylic acid, succinic acid, malic acid, ascorbic acid, benzoic acid, tannic acid, pamoic acid, alginic acid, polyglutamic acid, naphthalenesulfonic acid, naphthalenedisulfonic acid, polygalacturonic acid and the like. Examples of base salts include those derived from inorganic bases such as potassuim, sodium, lithium, ammonium, calcium, magnesium, iron, zinc, copper, manganese, aluminum and the like. Salts from derived from suitable organic non-toxic bases include salts of primary, secondary, and tertiary amines, substituted amines, cyclic amines, and basic ion exchange resins such as isopropylamine, trimethylamine, diethylamine, triethylamine, tripropylamine and ethanolamine.

The compounds can also be administered as pharmaceutically acceptable quaternary salts known by those skilled in the art, which specifically include the quaternary ammonium salt of the formula —NR⁺Z⁻, wherein R is hydrogen, alkyl, or benzyl, and Z is a counterion, including chloride, bromide, iodide, —O-alkyl, toluenesulfonate, methylsulfonate, sulfonate, phosphate, or carboxylate (such as benzoate, succinate, acetate, glycolate, maleate, malate, citrate, tartrate, ascorbate, benzoate, cinnamoate, mandeloate, benzyloate, and diphenylacetate). Moreover, the compounds of the invention can also be administered as prodrugs which can be converted to the active form in vivo.

The active compound is included in the pharmaceutically acceptable carrier or diluent in an amount sufficient to deliver to a patient a therapeutically effective amount without causing serious toxic effects in the patient treated. The compounds can be formulated in a variety of ways depending on the manner of administration. The concentration of the active compounds in these formulations can vary from 0.1 to 100% wt/wt. A preferred dose of the active compound for all of the above-mentioned conditions is in the range from about 0.01 to 550 mg/kg, preferably 300 to 550 mg/kg, more preferably 0.1 to 100 mg/kg per day, and more generally 0.5 to about 25 mg per kilogram body weight of the recipient per day. A typical topical dosage will range from 0.01-3% wt/wt in a suitable carrier. The effective dosage range of the pharmaceutically acceptable derivatives can be calculated based on the weight of the parent compound to be delivered. If the derivative exhibits activity in itself, the effective dosage can be estimated as above using the weight of the derivative, or by other means known to those skilled in the art.

When administered systemically, the ubiquitination inhibitor is preferably administered at a sufficient dosage to attain a blood level of the inhibitor from about 0.01 μM to about 100 μM, more preferably from about 0.05 μM to about 50 μM, still more preferably from about 0.1 μM to about 25 μM, and still yet more preferably from about 0.5 μM to about 20 μM. For localized administration, much lower concentrations than this may be effective, and much higher concentrations may be tolerated. One of skill in the art will appreciate that the dosage of ubiquitination inhibitor necessary to produce a therapeutic effect may vary considerably depending on the tissue, organ, or the particular animal or patient to be treated.

By “administration” is meant administering a therapeutically effective dose to a cell or patient. A therapeutically effective dose is a dose that produces the effects for which it is administered. The exact dose depends on the purpose of the treatment and can be ascertained by one skilled in the art using known techniques.

By “patient” is meant a human or other animal and organisms, for example, experimental animals. Thus, the compounds can be used for both human therapy and veterinary applications. In a preferred embodiment, the patient is human.

### Inhibition of Ubiquitination

In a third aspect, the invention provides a method of inhibiting ubiquitination in a cell, comprising contacting a cell in which inhibition of ubiquitination is desired with an inhibitor of ubiquitination of the invention.

Measurement of the ubiquitination can be achieved using known methodologies. (See, for example, WO 01/75145, US-2002-0042083-A1 and WO 03/076608, each of which is incorporated by reference in its entirety.)

Preferably, the method according to the third aspect of the invention causes an inhibition of cell proliferation of contacted cells. The phrase “inhibiting cell proliferation” is used to denote an ability of an inhibitor of ubiquitination to retard the growth of cells contacted with the inhibitor as compared to cells not contacted. An assessment of cell proliferation can be made by counting contacted and non-contacted cells using a Coulter Cell Counter (Coulter, Miami, Fla.), photographic analysis with Array Scan II (Cellomics) or a hemacytometer. Where the cells are in a solid growth (e.g., a solid tumor or organ), such an assessment of cell proliferation can be made by measuring the growth with calipers and comparing the size of the growth of contacted cells with non-contacted cells.

Preferably, growth of cells contacted with the inhibitor is retarded by at least 50% as compared to growth of non-contacted cells. More preferably, cell proliferation is inhibited by 100% (i.e., the contacted cells do not increase in number). Most preferably, the phrase “inhibiting cell proliferation” includes a reduction in the number or size of contacted cells, as compared to non-contacted cells. Thus, an inhibitor of ubiquitination according to the invention that inhibits cell proliferation in a contacted cell may induce the contacted cell to undergo growth retardation, to undergo growth arrest, to undergo programmed cell death (i.e., to apoptose), or to undergo necrotic cell death.

In some preferred embodiments, the contacted cell is a neoplastic cell. The term “neoplastic cell” is used to denote a cell that shows aberrant cell growth. Preferably, the aberrant cell growth of a neoplastic cell is increased cell growth. A neoplastic cell may be a hyperplastic cell, a cell that shows a lack of contact inhibition of growth in vitro, a benign tumor cell that is incapable of metastasis in vivo, or a cancer cell that is capable of metastasis in vivo and that may recur after attempted removal. The term “tumorigenesis” is used to denote the induction of cell proliferation that leads to the development of a neoplastic growth. In some embodiments, the ubiquitination inhibitor induces cell differentiation in the contacted cell. Thus, a neoplastic cell, when contacted with an inhibitor of ubiquitination may be induced to differentiate, resulting in the production of a non-neoplastic daughter cell that is phylogenetically more advanced than the contacted cell.

### Treatment for Cell Proliferative Diseases or Conditions

In some preferred embodiments, the contacted cell is in an animal. Thus, in a fourth aspect the invention provides a method for treating a cell proliferative disease or condition in an animal, comprising administering to an animal in need thereof an effective amount of an inhibitor of ubiquitination of the invention. Preferably, the animal is a mammal, more preferably a domesticated mammal. Most preferably, the animal is a human.

The term “cell proliferative disease or condition” is meant to refer to any condition characterized by aberrant cell growth, preferably abnormally increased cellular proliferation. Examples of such cell proliferative diseases or conditions include, but are not limited to, cancer, restenosis, and psoriasis. In particularly preferred embodiments, the invention provides a method for inhibiting neoplastic cell proliferation in an animal comprising administering to an animal having at least one neoplastic cell present in its body a therapeutically effective amount of a ubiquitination inhibitor of the invention. Most preferably, the invention provides a method for treating cancer comprising administering to a patient in need thereof an effective amount of an inhibitor of ubiquitination of the invention.

The term “therapeutically effective amount” is meant to denote a dosage sufficient to cause inhibition of ubiquitination in the cells of the subject, or a dosage sufficient to inhibit cell proliferation or to induce cell differentiation in the subject. Administration may be by any route, including, without limitation, parenteral, oral, sublingual, transdermal, topical, intranasal, intratracheal, or intrarectal. In certain particularly preferred embodiments, compounds of the invention are administered intravenously in a hospital setting. In certain other preferred embodiments, administration may preferably be by the oral route.

When administered systemically, the ubiquitination inhibitor is preferably administered at a sufficient dosage to attain a blood level of the inhibitor from about 0.01 μM to about 100 μM, more preferably from about 0.05 μM to about 50 μM, still more preferably from about 0.1 μM to about 25 μM, and still yet more preferably from about 0.5 μM to about 20 μM. For localized administration, much lower concentrations than this may be effective, and much higher concentrations may be tolerated. One of skill in the art will appreciate that the dosage of ubiquitination inhibitor necessary to produce a therapeutic effect may vary considerably depending on the tissue, organ, or the particular animal or patient to be treated.

### Biological Assay

The ubiquitination inhibition properties of compounds of the invention can be assayed by suitable methods that measure ubiquitin ligase activities. For example, methods that measure the ubiquitin ligase activities of MDM2 or APC2/APC11 can be used to assay the compounds of the invention.

### ASSAY EXAMPLE 1

### MDM2 Assay

The MDM2 assay used for measuring the attachment of ubiquitin to p53 was carried out as described in WO 01/75145 and WO 03/076608, each of which is incorporated by reference in its entirety. Briefly, Flag-ubiquitin was added to a solution containing GST-MDM2, E1, E2 and His-p53 and the reaction was carried out at 37° C. for 1 hr. After completion of the reaction, a sample of the solution was resolved by SDS-PAGE, analyzed by Western blot and the ligation of ubiquitin to p53 was measured by immunodetection of the ubiquitin-p53 complex using mouse anti-Flag and anti-mouse Ig-HRP.

The MDM2 assay was also carried out in Nickel-substrate 96-well plates using His-tagged p53. In this method, Flag-ubiquitin was added to a solution containing MDM2, E1, E2 and His-p53 and the reaction was carried out at room temperature for 1 hr. After the reaction was completed, the wells were washed with PBS and to each well was added mouse anti-lag and anti-mouse Ig-HRP. The plates were then incubated for 1 hour and then washed again with PBS to remove excess antibodies. Luminol was then added to each well and the ligation of ubiquitin to p53 was measured by luminescence to detect the ubiquitin-p53 complex. The compounds to be assayed were dissolved in DMSO and added before the addition of Flag-ubiquitin. Activity in the presence of the compound was determined relative to a parallel control in which only DMSO was added. Values of the IC50 were typically determined using different concentrations of the compound, although as few as 2 concentrations may be used to approximate the IC50 value.

### ASSAY EXAMPLE 2

### APC-11/APC-2 Ligase Assay

E3 (His-APC11/APC2—“APC”) auto-ubiquitination was measured as described in U.S. patent application Ser. No. 09/826,312 (Publication No. US-2002-0042083-A1), which is incorporated by reference in its entirety. Details of the protocol are described below. Activity in the presence of the compound was determined relative to a parallel control in which only DMSO was added. Values of the IC₅₀ were typically determined using 6 or 8 different concentrations of the compound, although as few as 2 concentrations may be used to approximate the IC₅₀ value.

Nickel-coated 96-well plates (Pierce 15242) were blocked for 1 hour with 100 μl of blocking buffer at room temperature. The plates were washed 4 times with 225 μl of 1×PBS and 80 μl of the reaction buffer were added that contained 100 ng/well of Flag-ubiquitin. To this, 10 μl of the test compound diluted in DMSO were added. After the test compound was added, 10 μl of E1 (human), E2 (Ubch5c), and APC in Protein Buffer was added to obtain a final concentration of 5 ng/well of E1, 20 ng/well of E2 and 100 ng/well of APC. The plates were shaken for 10 minutes and incubated at room temperature for 1 hour. After incubation, the plates were washed 4 times with 225 μl of 1×PBS and 100 μl/well of Antibody Mix were added to each well. The plates were incubated at room temperature for another hour after which they were washed 4 times with 225 μl of 1×PBS and 100 μl/well of Lumino substrate were added to each well. The luminescence was measured by using a BMG luminescence microplate reader.

To prepare the Blocking Buffer (1 liter; 1% Casein in 1×PBS), 10 grams of Casein (Hammersten Grade Casein from Gallard-Schlesinger Inc. #440203) were placed into 1 liter of 1×PBS, stirred on a hot plate and kept between 50-60° C. for an hour. The buffer was allowed to cool to room temperature and then filtered using a Buchner Funnel (Buchner filter funnel 83 mm 30310-109) and Whatman filter paper (Whatman Grade No.1 Filter paper 28450-070). It was stored at 4° C. until used.

The reaction buffer consisted of 62.5 mM Tris pH 7.6 (Trizma Base—Sigma T-8524), 3 mM MgCl₂ (Magnesium Chloride—Sigma M-2393), 1 mM DTT (Sigma D-9779), 2.5 mM ATP (Roche Boehringer Mann Corp. 635-316), 100 ng/well of Flag-ubiquitin, 0.1% BSA (Sigma A-7906), and 0.05% Tween-20 (Sigma P-7949).

The Protein Buffer consisted of 20 mM Tris pH 7.6, 10% glycerol (Sigma G-5516) and 1 mM DTT.

The antibody mix consisted of 0.25% BSA (Sigma A-7906) in 1×PBS, 1/50,000 anti-Flag (Sigma F-3165), 1/100,000 of anti-Mouse IgG-HRP (Jackson Immunoresearch #115-035-146).

The substrate mix consisted of SuperSignal Substrate from Pierce (catalog number 37070ZZ) and was prepared by mixing 100 ml of the peroxide solution, 100 ml of the enhancer solution and 100 ml of Milli-Q® water.

A second ubiquitin assay was performed substantially as described above, with a few Modifications. No nickel substrate was used in the reaction wells, so all of the components were free in solution. Equal amounts of fluorescein labeled ubiquitin moiety and labeled ubiquitin moiety were used. The reaction was performed at room temperature for 2 hours in a volume of 100-150 μl, then stopped with 50 μl of 0.5M EDTA, pH 8.

Following the reaction, the products were separated in PBS with 1 mM TCEP by HPLC on a Superdex-75 HR 10/30 size-exclusion column using fluorescence emission detection. A larger molecular weight cutoff gel-filtration column (e.g., Superdex 200 HR 10/30) could be used to resolve individual ligation species.

Table 1 below lists representative IC₅₀ values of the compounds of the invention determined by the assays described above. Whereas each compound recited in the table below was presented above as a specific geometric isomer (i.e., 5E or 5Z), it is expected that the compounds tested to generate the data in the table below were a mixture of the 5E and 5Z geometric isomers.

| | LIGASE E3 | LIGASE E3 | |
|---|---|---|---|
| | APC2/APC11 | APC2/APC11 | LIGASE_E3 |
| Cpd | H E1 | H E1/GEL | MDM2/P53 |
| 1 | 2, 4 | 2, 20 | |
| 4 | 9999 | | |
| 23 | 5.15 | | 7.94 |
| 25 | 2.06 | | 5.64 |
| 27 | | | 10.68 |
| 28 | | | 0.8 |
| 29 | 1.44 | | 1.21 |
| 30 | 5.49 | | 2.32 |
| 33 | 9999 | | 6.1 |
| 40 | | | 5.52 |
| 45 | | | 9999 |
| 46 | 0.91 | | 4.92 |
| 50 | 3.7 | | 2.69 |
| 51 | 12.1 | | 9.24 |
| 54 | 0.035 | | 0.074 |
| 55 | 0.15, 2.5, 5 | 4, 10 | |
| 63 | 0.2 | | |
| 65 | 9999 | | 100 |
| 69 | | | 14.24 |
| 75 | | | 13.63 |
| 77 | | | 0.81 |
| 82 | 5 | | 1.5 |
| 96 | 10 | | 0.56 |
| 98 | | | 11.59 |
| 99 | | | 0.21 |
| 103 | 0.3, 0.5 | 0.6, 2 | 3.87 |
| 116 | 5 | | |
| 145 | 10 | | |
| 146 | 10 | | |
| 148 | 0.3, 0.4 | 1, 2 | |
| 153 | 3, 20 | 2 | |
| 160 | 1 | 0.6 | |
| 172 | 2 | 2 | |
| 174 | 20 | | |
| 175 | 1.5 | 1, 2 | |
| 176 | 0.4, 0.7 | 0.6, 10 | |
| 177 | 0.4 | 2, 20 | |
| 183 | 0.1 | | |
| 199 | 0.1 | | |
| 238 | 5 | | |
| 244 | 9999 | | |
| 254 | | | 24.84 |
| 256 | | | 0.53 |
| 269 | 20 | | |
| 270 | 3 | 9999 | |
| 271 | 20 | | |
| 272 | | | |
| 274 | 0.11 | | 1.18 |
| 275 | 0.32 | | 1.16 |
| 278 | | | 5.21 |
| 280 | 0.56 | | 0.25 |
| 299 | 9999 | | 0.45 |
| 301 | 0.41 | | 0.93 |
| 302 | | | 4.6 |
| 305 | | | 10.82 |

### ASSAY EXAMPLE 3

### ROC1/CUL1 Ubiquitin Ligase Assay

Inhibition of ubiquitin ligase activity of E1+E2+E3 was measured using the protocol as described in WO 01/75145 with E3 as the ROC1/CUL1, ROC1/CUL2, or ROC2/CUL5 complex.

Materials and Methods

The wells of nickel-substrate 96-well plates (Pierce Chemical) are blocked with 100 μl of 1 casein/phosphate buffered saline (PBS) for 1 hour at room temperature, then washed with 200 μl of PBST (0.1% Tween-20 in PBS) 3 times. To each well is added the following Flag-ubiquitin (see above) reaction solution: 62.5 mM Tris pH 7.5, 6.25 mm MgCl₂, 0.75 mM DTT, 2.5 mM ATP, 2.5 mM NaF1, 2.5 nM Okadaic acid, 100 ng Flag-ubiquitin (made as described above).

The buffer solution is brought to a final volume of 80 μl with Milipore-filtered water, followed by the addition of 10 μl DMSO.

To the above solution is then added 10 μl of ubiquitination enzymes in 20 mM Tris buffer, pH 7.5, and 5% glycerol. E2-Ubch5c and E3-His ROC1/Cul1, ROC1/CUL2, and ROC2/CUL5 are made as described in WO 01/75145. E1 is obtained commercially (Affiniti Research Products, Exeter, U. K.). The following amounts of each enzyme are used for these assays: 5 ng/well of E1; 25 nl/well E2; and 100 ng/well His-E3. Varying amounts of compounds according to the invention are added and the reaction allowed to proceed at room temperature for 1 hour.

Following the ubiquitination reaction, the wells are washed with 200 μl of PBST 3 times. For measurement of the enzyme-bound ubiquitin, 100 gel of Mouse anti-Flag (1:10,000) and anti-Mouse Ig-HRP (1:15,000) in PBST are added to each well and allowed to incubate at room temperature for 1 hour. The wells are then washed with 200 μl of PBST 3 times, followed by the addition of 100 μl of luminol substrate (1/5 dilution). Luminescence for each well is then measured using a fluorimeter.

Compound 284 was found to have a ROC1/CUL1 IC₅₀ of 800 nM, a ROC1/CUL2 IC₅₀ of 800 nM, and a ROC2/CUL5 IC₅₀ of 200 nM. Compound 304 was found to have a ROC1/CUL1 IC₅₀ of 1 μM, a ROC1/CUL2 IC₅₀ of 1 μM, and a ROC2/CUL5 IC₅₀ of 800 nM.

1. A composition comprising a compound of the formula 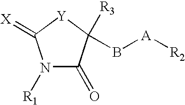
or pharmaceutically acceptable salts thereof together with a pharmaceutically acceptable carrier, excipient, or diluent, wherein
A is aryl or heteroaryl;
B is C₁-C₆ alkyl or C₂-C₆ alkenyl;
X is sulfur, oxygen, ═CR₄R₅, ═NR₄, ═NC(O)R₄, or ═NSO₂R₄,
Y is sulfur, oxygen, —C(R₄)(R₅)—, —N(R₄)—, —NC(O)(R₄)—, —NSO₂(R₄)—, —S(O)₂—, or —S(O)—;
R₁ is —H, —NH₂, C₁-C₆ alkyl, C₁-C₂ alkenyl, C₁-C₆ alkyl-S—C₁-C₆ alkyl, C₀-C₆ alky-aryl, C₀-C₆ alkyl-C(O)OR₆, C₀-C₆ alkyl-heteroaryl, C₀-C₆ alkyl-heterocyclyl, C₀-C₆ alkyl-carbocyclyl, —NH—SO₂-aryl, —C₀-C₆ alkyl-C(O)NR₆R₇, —C₀-C₆ alkyl-C(S)NR₆R₇, C₀-C₆ alky-heteroaryl-aryl, —NHC(O)-aryl, C₀-C₆ alkyl-C(O)NH-C₀-C₆ alkyl-C(O)—O—R₆, C₀-C₆ alkyl-C(O)—NH—C₀-C₆ alkyl-aryl, C₀-C₆ alkyl-C(O)—NH—C₀-C₆ alkyl-heteroaryl, C₀-C₆ alkyl-C(O)—NH—C₀-C₆ alkyl-heterocyclyl, C₀-C₆ alkyl-C(O)—NH—C₀-C₆ alkyl-carbocyclyl, —SO₂—R₆, C(O)—R₆ or —C(O)—OR₆, wherein each one of the alkyl, aryl, heteroaryl, heterocyclic and carbocyclyl are optionally substituted with one or more R₅;
R₂ is —H, halogen, C₁-C₆ alkyl, C₀-C₆ alky-aryl, —NO₂, C₀-C₆ alkyl-C(O)—OR₆, C₀-C₆ alkyl-heteroaryl, C₀-C₆ alkyl-heterocyclyl, C₀-C₆ alkyl-carbocyclyl, —N(R₆)—C(O)NR₆R₇, —NHSO₂-aryl, C₀-C₆ alky-heteroaryl-aryl or —C(O)—R₆, wherein each one of the aryl, heteroaryl, heterocyclic and carbocyclyl are optionally substituted with one or more R₄;
R₃ is —H, C₁-C₆ alkyl or C₂-C₆ alkenyl; or
R₃ and B together with the carbon atom to which they are attached form an alkenyl or a spirocyclic ring;
R₄ is halogen, oxo, —C(O)OR₆, —NO₂, C₁-C₆ alkyl optionally substituted with halo, —C₁-C₆ alkoxy optionally substituted with halo, —CH₃, —SO₂NH₂ or —C(O)—OR₆;
R₅ is halogen, oxo, C₁-C₆ alkoxy, C₁-C₆ alkyl, C₀-C₆ alkyl-aryl, —NO₂, di(C₁-C₆ alkyl)amino, —CF₃, —OH, —SO₂NH₂ or —C(O)—OR₆; and
R₆ and R₇ are independently —H, halogen, C₁-C₆ alkoxy, C₁-C₆ alkyl, C₂-C₆ alkenyl, aryl, di(C₁-C₆ alkyl)amino, —CF₃, —OH or —C(O)—OR₆.

2. The composition according to claim 1 wherein the compound is of the formula

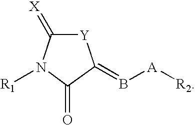

3. The composition according to claim 2 wherein the compound is of the formula

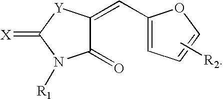

4. The composition according to claim 3 wherein R₁ is —H, C₁-C₆ alkyl, C₁-C₂ alkenyl, C₀-C₆ alky-aryl, C₀-C₆ alkyl-C(O)OR₆, C₀-C₆ alkyl-heteroaryl, C₀-C₆ alkyl-heterocyclyl, C₀-C₆ alkyl-carbocyclyl or C₀-C₆ alky-heteroaryl-aryl, and R₂ is —H, halogen, C₁-C₆ alkyl, C₀-C₆ alky-aryl.

5. The composition according to claim 4 wherein R₁ is —H, C₁-C₆ alkyl, C₁-C₂ alkenyl, C₀-C₆ alky-aryl, or C₀-C₆ alkyl-C(O)OR₆ and R₂ is C₀-C₆ alky-aryl.

6. The composition according to claim 5 wherein R₁ is —H, allyl, phenyl or benzyl and R₂ is phenyl.

7. The composition according to claim 3 wherein the compound is of the formula

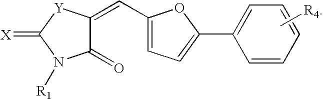

8. The composition according to claim 7 wherein R₁ is —H, C₁-C₆ alkyl, C₁-C₂ alkenyl, C₀-C₆ alky-aryl, C₀-C₆ alkyl-C(O)OR₆, C₀-C₆ alkyl-heteroaryl, C₀-C₆ alkyl-heterocyclyl, C₀-C₆ alkyl-carbocyclyl or C₀-C₆ alky-heteroaryl-aryl, and R₄ is halogen, oxo, —NO₂, C₁-C₆ alkyl, —C₁-C₆ alkoxy, —CF₃, —SO₂NH₂, or —C(O)—OR₆.

9. The composition according to claim 8 wherein R₁ is —H, C₁-C₆ alkyl, C₁-C₂ alkenyl, C₀-C₆ alky-aryl, or C₀-C₆ alkyl-C(O)OR₆, and R₄ is halogen, —NO₂, C₁-C₆ alkyl, —C₁-C₆ alkoxy, —CF₃, —SO₂NH₂, or —C(O)—OR₆.

10. The composition according to claim 9 wherein R₁ is —H, allyl, phenyl or benzyl and R₄ is chloro, bromo, fluoro, —NO₂, —OCH₃, —CF₃ or —C(O)—OH.

11. A compound of the formula 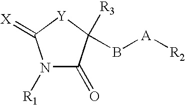
or pharmaceutically acceptable salts thereof together with a pharmaceutically acceptable carrier, excipient, or diluent, wherein
A is aryl or heteroaryl;
B is C₁-C₆ alkyl or C₂-C₆ alkenyl;
X is sulfur, oxygen, ═CR₄R₅, ═NR₄, ═NC(O)R₄, or ═NSO₂R₄,
Y is sulfur, oxygen, —C(R₄)(R₅)—, —N(R₄)—, —NC(O)(R₄)—, —NSO₂(R₄)—, —S(O)₂—, or —S(O)—;
R₁ is —H, —NH₂, C₁-C₆ alkyl, C₁-C₂ alkenyl, C₁-C₆ alkyl-S—C₁-C₆ alkyl, C₀-C₆ alky-aryl, C₀-C₆ alkyl-C(O)OR₆, C₀-C₆ alkyl-heteroaryl, C₀-C₆ alkyl-heterocyclyl, C₀-C₆ alkyl-carbocyclyl, —NH—SO₂-aryl, —C₀-C₆ alkyl-C(O)NR₆R₇, —C₀-C₆ alkyl-C(S)NR₆R₇, C₀-C₆ alky-heteroaryl-aryl, —NHC(O)-aryl, C₀-C₆ alkyl-C(O)NH—C₀-C₆ alkyl-C(O)—O—R₆, C₀-C₆ alkyl-C(O)—NH—C₀-C₆ alkyl-aryl, C₀-C₆ alkyl-C(O)—NH—C₀-C₆ alkyl-heteroaryl, C₀-C₆ alkyl-C(O)NH—C₀-C₆ alkyl-heterocyclyl, C₁-C₆ alkyl-C(O)—NH—C₀-C₆ alkyl-carbocyclyl, —SO₂—R₆, C(O)—R₆ or —C(O)—OR₆, wherein each one of the alkyl, aryl, heteroaryl, heterocyclic and carbocyclyl are optionally substituted with one or more R₅;
R₂ is —H, halogen, C₁-C₆ alkyl, C₀-C₆ alky-aryl, —NO₂, C₀-C₆ alkyl-C(O)—OR₆, C₀-C₆ alkyl-heteroaryl, C₀-C₆ alkyl-heterocyclyl, C₀-C₆ alkyl-carbocyclyl, —N(R₆)—C(O)NR₆R₇, —NHSO₂-aryl, C₀-C₆ alky-heteroaryl-aryl or —C(O)—R₆, wherein each one of the aryl, heteroaryl, heterocyclic and carbocyclyl are optionally substituted with one or more R₄;
R₃ is —H, C₁-C₆ alkyl or C₂-C₆ alkenyl; or
R₃ and B together with the carbon atom to which they are attached form an alkenyl or a spirocyclic ring;
R₄ is halogen, oxo, —C(O)OR₆, —NO₂, C₁-C₆ alkyl optionally substituted with halo, —C₁-C₆ alkoxy optionally substituted with halo, —CF₃, —SO₂NH₂ or —C(O)—OR₆;
R₅ is halogen, oxo, C₁-C₆ alkoxy, C₁-C₆ alkyl, C₀-C₆ alkyl-aryl, —NO₂, di(C₁-C₆ alkyl)amino, —CF₃, —OH, —SO₂NH₂ or —C(O)—OR₆; and
R₆ and R₇ are independently —H, halogen, C₁-C₆ alkoxy, C₁-C₆ alkyl, C₂-C₆ alkenyl, aryl, di(C₁-C₆ alkyl)amino, —CF₃, —OH or —C(O)—OR₆,
provided the compound is not a compound of the formula 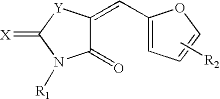
X and Y are independently sulfur, oxygen, —CR₄R₅, —NR₄, —NC(O)R₄, —NSO₂R₄, —SO₂, or —SO;
R₁ is —H, —NH₂, C₁-C₆ alkyl, C₁-C₂ alkenyl, C₁-C₆ alkyl-S—C₁-C₆ alkyl, C₀-C₆ alky-aryl, C₀-C₆ alkyl-C(O)OR₆, C₀-C₆ alkyl-heteroaryl, C₀-C₆ alkyl-heterocyclyl, C₀-C₆ alkyl-carbocyclyl, —NH—SO₂-aryl, —C₀-C₆ alkyl-C(O)NR₆R₇, —C₀-C₆ alkyl-C(S)NR₆R₇, C₀-C₆ alky-heteroaryl-aryl, —NHC(O)-aryl, C₀-C₆ alkyl-C(O)NH—C₀-C₆ alkyl-C(O)—O—R₆, C₀-C₆ alkyl-C(O)—NH—C₀-C₆ alkyl-aryl, C₀-C₆ alkyl-C(O)—NH—C₀-C₆ alkyl-heteroaryl, C₀-C₆ alkyl-C(O)—NH—C₀-C₆ alkyl-heterocyclyl, C₀-C₆ alkyl-C(O)—NH—C₀-C₆ alkyl-carbocyclyl, —SO₂—R₆, C(O)—R₆, or —C(O)—OR₆, wherein each one of the alkyl, aryl, heteroaryl, heterocyclic and carbocyclyl are optionally substituted with one or more R₅;
R₂ is —H, halogen, C₁-C₆ alkyl, C₀-C₆ alky-aryl, —NO₂, C₀-C₆ alkyl-C(O)—OR₆, C₀-C₆ alkyl-heteroaryl, C₀-C₆ alkyl-heterocyclyl, C₀-C₆ alkyl-carbocyclyl, —N(R₆)—C(O)NR₆R₇, —NHSO₂-aryl, C₀-C₆ alky-heteroaryl-aryl, or —C(O)—R₆, wherein each one of the aryl, heteroaryl, heterocyclic and carbocyclyl are optionally substituted with one or more R₄;
R₄ is halogen, oxo, —C(O)OR₆, —NO₂, C₁-C₆ alkyl optionally substituted with halo, —C₁-C₆ alkoxy optionally substituted with halo, —CF₃, —SO₂NH₂, or —C(O)—OR₆;
R₅ is halogen, oxo, C₁-C₆ alkoxy, C₁-C₆ alkyl, C₀-C₆ alkyl-aryl, —NO₂, di(C₁-C₆ alkyl)amino, —CF₃, —OH, —SO₂NH₂, or —C(O)—OR₆; and
R₆ and R₇ are independently —H, halogen, C₁-C₆ alkoxy, C₁-C₆ alkyl, C₂-C₆ alkenyl, aryl, di(C₁-C₆ alkyl)amino, —CF₃, —OH, or —C(O)—OR₆.

12. A method of inhibiting ubiquitination in a cell comprising contacting a cell in which inhibition of ubiquitination is desired with a composition according to claim 1.

13. The method according to claim 12 wherein the cell is from a mammal.

14. The method according to claim 13 wherein the mammal is human.

15. A method of treating cell proliferative diseases or conditions comprising administering to a patient an effective amount of a composition according to claim 1.

16. The method according to claim 15 wherein the cell proliferative diseases are cancers.

17. The method according to claim 16 wherein the patient is human.

18. A method of inhibiting ubiquitination in a cell comprising contacting a cell in which inhibition of ubiquitination is desired with a composition according to claim 2.

19. A method of inhibiting ubiquitination in a cell comprising contacting a cell in which inhibition of ubiquitination is desired with a composition according to claim 3.

20. A method of inhibiting ubiquitination in a cell comprising contacting a cell in which inhibition of ubiquitination is desired with a composition according to claim 7.
